(19) United States
(12) Patent Application Publication
(10) Pub. No.: US 2016/0022826 A1
(43) Pub. Date: Jan. 28, 2016

(54) CARBOHYDRATE-MODIFIED GLYCOPROTEINS AND USES THEREOF

(71) Applicant: NEWLINK GENETICS CORPORATION, Ames, IA (US)

(72) Inventors: Wenlan Alex CHEN, Ames, IA (US); Mario R. MAUTINO, Ames, IA (US); Brian MARTIN, Ames, IA (US)

(21) Appl. No.: 14/775,841

(22) PCT Filed: Mar. 13, 2014
(86) PCT No.: PCT/US14/25702

§ 371 Date: Sep. 14, 2015

Related U.S. Application Data
(60) Provisional application No. 61/800,623, filed on Mar. 15, 2013.

(51) Int. Cl. A61K 47/48; A61K 39/145; C12N 7/00; A61K 39/39

(52) U.S. Cl. CPC A61K 47/48092; A61K 47/48261; A61K 47/4833; A61K 39/39; A61K 39/145; C12N 7/00; C12N 2760/16134; A61K 2039/5258

(57) ABSTRACT

The present invention provides immunogenic compounds which stimulate immune responses in a subject. The present invention provides compositions comprising an isolated glycoprotein antigen covalently bound at pre-existing carbohydrate residues present on the glycoprotein to a carbohydrate epitope. The present invention also provides a method to induce an immune response in a subject comprising administering the compounds of the invention. The present invention further provides methods of making the compounds of the invention and methods of using the compounds of the invention to stimulate immune responses to infectious disease agents and tumors.

### CROSS REFERENCE TO RELATED APPLICATIONS

This application claims priority to U.S. Provisional Application No. 61/800,623, filed Mar. 15, 2013 which is incorporated by reference herein in its entirety.

### FIELD OF THE INVENTION

The present invention relates to compounds which stimulate immune responses in a subject. In particular, the present invention provides compositions comprising an isolated carbohydrate epitope covalently bound at pre-existing carbohydrate residues present on a glycoprotein. The invention further provides methods of making the compounds of the invention. The present invention also provides a method to induce an immune response in a subject comprising administering the compounds of the invention. The present invention is also directed to methods of using the compounds of the invention to stimulate immune responses to infectious disease agents and tumors.

### BACKGROUND OF THE INVENTION

The targeting of autologous vaccines towards antigen presenting cells (APC) via the in vivo complexing between carbohydrate epitopes and antibodies that recognize such carbohydrate epitopes presents a promising avenue of eliciting a robust immune response to both treat and to immunize against infectious disease and tumors.

Several strategies have been developed to improve the immunogenicity of polypeptide antigens. Modification of the amino acid sequence of epitopes can improve the efficacy of vaccines by: 1) increasing affinity of peptide for MHC molecules (Berzofsky 1993; Berzofsky et al. 2001; Rosenberg et al. 1998a); 2) increasing binding to the TCR (Fong et al. 2001; Rivoltini et al. 1999; Zaremba et al. 1997); or 3) inhibiting proteolysis of the peptide by serum peptidases (Berzofsky et al. 2001; Parmiani et al. 2002). Epitope enhancement has shown efficacy in clinical trials (Rosenberg et al. 1998a), however, this is a laborious process that is specific for each epitope/MHC pair evaluated. Furthermore, these vaccines often require combinations with potent adjuvants and stimulating cytokines.

Vaccination with purified antigens in the form of soluble polypeptides results in uptake of these antigens by pinocytosis, endocytocis or phagocytosis through the endosomal-lysosomal pathway, which ultimately delivers peptide onto surface MHC class II but not to MHC class I complexes. Thereby, vaccination with soluble polypeptides in their native form does result mainly in a CD4+ mediated immune response but not in a potent stimulation of CD8+ T cells, which is believed to be the main T cell type needed for an efficient immune response against tumors. It has been demonstrated that uptake of antigen-antibody immunocomplexes by the FcγRI and FcγRIII receptors in DCs mediates activation and maturation of DCs and promotes cross-presentation of antigen in the context of both MHC class I and class II complexes, thereby stimulating both CD4+ and CD8+ cells (Ackerman et al. 2005; Heath et al. 2004; Heath and Carbone 2001; Palliser et al. 2005; Rafiq et al. 2002; Schnurr et al. 2005). Consistently with this, vaccination of mice with DCs loaded with immunocomplexes elicits a protective antitumor response against tumors bearing the antigen present in the immunocomplex (Rafiq et al. 2002). It is important to highlight, however, that in this study the animals did not have a pre-existing state of immunotolerance against the vaccinating antigen.

An efficient way to promote the formation of immunocomplexes in vivo is by modifying the antigen to contain epitopes or mimotopes against which the recipient host has naturally occurring pre-existing antibodies. This can be accomplished by several means such as by introducing A or B blood antigen groups and administering the modified antigen to an O-type blood recipient. Alternatively, a preferred method is to modify the antigen to contain carbohydrate epitopes, such as the αGal, L-Rhamnose, or Forssman disaccharide epitopes, that are recognized by natural antibodies existing in humans.

It has been demonstrated that immunogenicity of viral or xenogeneic proteins, against which there is no pre-established tolerance, is enhanced by introduction of αGal epitopes. For example, immunization of αGalactosyl(1,3)transferase (αGT)-knockout mice with BSA conjugated with αGal led to significant production of anti-BSA IgG antibodies without the need for adjuvant. The presence of αGal also led to an increase in the T cell response to BSA (Benatuil et al. 2005). Additionally, it has been shown that the presence of anti-αGal antibodies enhanced the cytotoxic T cell response against a viral antigen following vaccination with MoMLV transformed cell lines that express αGal on their surface (Benatuil et al. 2005). Similarly, enzymatic modification of influenza hemagglutinin with recombinant αGT results in addition of αGT epitopes to HA. It has been shown that αGal⁽⁺⁾ HA present in whole virions increases the uptake and T cell stimulating capacity of antigen presenting cells, which is reflected by increased proliferation of a HA-specific T cell clone (Galili et al. 1996). This indicates that the presence of αGal epitopes in conjunction with anti-αGal antibodies can provide an adjuvant effect that allows for efficient T cell and B cell priming to native protein antigens that do not bear αGal epitopes. In these previous experiments, the αGT KO hosts did not have a pre-existing state of immune tolerance against the αGal⁽⁺⁾ antigens and were induced to develop anti-αGal antibodies by immunization with pig kidney membranes or rabbit red blood cells, which bear the αGal antigen.

In the experiments mentioned above, modification of recombinant proteins to introduce αGal was achieved by treatment of the glycoprotein antigens (purified HA or HIV-1 gp120) with recombinant αGT and UDP-Gal. This technology has several disadvantages: i) recombinant αGT is unstable and prone to deactivation; ii) it is difficult to obtain sufficient amounts of recombinant or purified αGT to satisfy real clinical demand of the vaccines produced; and iii) αGT has to be separated from the final vaccine product.

An alternative to enzymatic modification is to add the αGal epitope to the target vaccine protein by chemical modification using activated cross-linkers.

The most common current cross-linking approach binds the carbohydrate epitope to thiol groups on cysteine or to amino groups of lysine residues on the glycoprotein antigen. The N-hydroxysuccinimide ester (NHS) readily reacts with amino group of lysine residues under physiological conditions. Similarly, maleimide reacts with the thiol group of cysteine. Therefore, NHS or maleimide activated carbohydrate epitope linkers (including αGal, rhamnose, and Forssman disaccharide) are currently used. This type of modification efficiently binds carbohydrate antigens to lysines or cysteines on the protein target. However, due to the fact that the reaction between NHS and the amino group of lysine or the maleimide group on cysteines generates a type of covalent bond that is not present in nature, these modified proteins cannot be normally deglycosylated during antigen processing by the N- and O-glycosidases present in the lysosomes of the antigen presenting cells. Consequently, the peptides derived from antigen processing will still bear the carbohydrate-linker modification which will prevent the efficient binding of such peptides to the major histocompatibility molecules for antigen presentation. Moreover, since most of the lysines are easily modified, due to the large number of lysines exposed on the protein's surfaces this strategy may cause the blockage of antigenic regions thus the complex will not elicit the desired immune response. Furthermore, too many modifications on the glycoprotein antigen backbone can result in a change in protein conformation and consequently reduce and/or destroy the protein's biological activity.

In order to overcome these disadvantages, a more site-specific and selective modification strategy that allows for in vivo immunocomplex formation with the vaccinated glycoprotein-antigen, FcγR-mediated antigen uptake, removal of the glycan modification during antigen processing, and peptide antigen presentation in the context of both MHC-I and MHC-II complexes is desired.

### SUMMARY OF THE INVENTION

The present invention provides compositions which will stimulate an immune response in a subject, comprising a carbohydrate epitope covalently bound to pre-existing carbohydrate residues present on a glycoprotein antigen. Addition of a carbohydrate epitope such as the αGal, L-Rhamnose, or Forssman epitopes, to a glycoprotein antigen triggers the in vivo formation of immunocomplexes between the complexed antigen and natural anti-carbohydrate epitope antibodies. Modification of glycoprotein antigens with a carbohydrate epitope increases their immunogenicity, thereby eliciting a humoral and cellular immune response against the unmodified antigen present in a subject. The present invention also provides a method to induce an immune response in a subject comprising administering the compounds of the invention. The invention further provides methods of making the compounds of the invention.

In one aspect of the invention, immune adjuvant compounds are provided. In one embodiment, the immune adjuvant compounds comprise a chemical structure of Su-O—R₁—ONH₂, wherein Su is any saccharide, for example, a monosaccharide, disaccharide, trisaccharide, tetrasaccharide or other polysaccharide to which humans have natural or acquired pre-existing antibodies, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups. In a further embodiment, Su is an αGal, L-Rhamnose, or Forssman epitope. In a further embodiment, the αGal epitope has the structure Gal(α1-3)Gal(β1-4)Glc or Gal(α1-3)Gal(β1-4)GlcNAc.

In another aspect of the invention, isolated antigens are provided. In one embodiment, the isolated antigen comprises a modified glycoprotein having a carbohydrate epitope covalently bound at a carbohydrate and amino acid residue on the glycoprotein antigen. In another embodiment, the carbohydrate epitope is a monosaccharide, disaccharide, trisaccharide, tetrasaccharide, or pentasaccharide to which humans have natural or acquired pre-existing antibodies. In another embodiment, the carbohydrate epitope is bound to the carbohydrate and amino acid residue on the glycoprotein via a linker. In another embodiment, the carbohydrate-linked glycoprotein has the structure Su-O—R₁—O—N=GP, wherein R₁ is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups and wherein said N is double bonded to the carbohydrate and amino acid residue on said glycoprotein.

In one embodiment, the invention provides an isolated antigen comprising a modified glycoprotein having the structure Su-O—R₁—O—N═CR, wherein Su is a monosaccharide, disaccharide, trisaccharide, tetrasaccharide or pentasaccharide, and wherein CR represents the carbohydrate residue of said glycoprotein which is bound to N through an oxime bond, and wherein R₁ is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups.

In one embodiment, the isolated antigen comprises a modified glycoprotein wherein one or more carbohydrate residues in said glycoprotein have been chemically modified with an immune adjuvant compound comprising a chemical structure Su-O—R₁—ONH₂, wherein Su is any saccharide, for example, a monosaccharide, disaccharide, trisaccharide, tetrasaccharide or other polysaccharide to which humans have natural or acquired pre-existing antibodies, and wherein R₁ is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups. In a further embodiment, Su is an αGal, L-Rhamnose, or Forssman epitope. In a further embodiment, the αGal epitope has the structure Gal(α1-3)Gal(β1-4)Glc or Gal(α1-3)Gal(β1-4)GlcNAc.

In another aspect of the invention, a pharmaceutical composition useful to elicit an immune response is provided. In one embodiment, the pharmaceutical composition comprises an isolated antigen comprising a modified glycoprotein wherein one or more carbohydrate residues in said glycoprotein have been chemically modified with an immune adjuvant compound comprising a chemical structure Su-O—R₁—ONH₂, wherein Su is a monosaccharide, disaccharide, trisaccharide, tetrasaccharide or pentasaccharide to which humans have natural or acquired pre-existing antibodies, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups and a carrier. In a further embodiment, Su is an αGal, L-Rhamnose, or Forssman epitope. In a further embodiment, the αGal epitope has the structure Gal(α1-3)Gal(β1-4)Glc or Gal(α1-3)Gal(β1-4)GlcNAc.

In another aspect of the invention, a method to induce an immune response in a subject is provided. In one embodiment, the method comprises administering to said subject an effective amount of an isolated antigen comprising a modified glycoprotein wherein one or more carbohydrate residues in said glycoprotein have been chemically modified with an immune adjuvant compound comprising a chemical structure Su-O—R₁—ONH₂, wherein Su is a monosaccharide, disaccharide, trisaccharide, tetrasaccharide or pentasaccharide to which humans have natural or acquired pre-existing antibodies, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups and a carrier. In a further embodiment, Su is an αGal, L-Rhamnose, or Forssman epitope. In a further embodiment, the αGal epitope has the structure Gal(α1-3)Gal(β1-4)Glc or Gal(α1-3)Gal(β1-4)GlcNAc. In a further embodiment, the subject is human.

In another aspect of the invention, a method to produce the isolated antigens of the invention is provided. In one embodiment, the method to produce an isolated antigen comprising a modified glycoprotein wherein one or more carbohydrate residues in said glycoprotein have been chemically modified with an immune adjuvant compound comprising a chemical structure Su-O—R₁—ONH₂, wherein Su is a monosaccharide, disaccharide, trisaccharide, tetrasaccharide or pentasaccharide to which humans have natural or acquired pre-existing antibodies, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups, by reacting said immune adjuvant compound with said glycoprotein to selectively attach said immune adjuvant compound to an oxidized carbohydrate residue present in said glycoprotein.

In one embodiment of the present invention, the isolated antigens are produced by oxidizing a carbohydrate on said glycoprotein to produce a reactive carbonyl group, and reacting said carbonyl group with the aminooxy group on said immune adjuvant compound to form an oxime bond and generate said isolated antigen. In another embodiment, said oxidizing step is performed using an oxidant selected from the group consisting of NaIO₄, galactose oxidase, or an engineered variant thereof. In a further embodiment, said galactose oxidase or engineered variant thereof is free or immobilized. In yet a further embodiment, said glycoprotein lacks a terminal galactose or N-acetylgalactosamine or sialic acid. In a further embodiment said glycoprotein comprises an aldehyde group.

In another aspect, the invention provides for isolated antigens. In one embodiment, the isolated antigen comprises an immune adjuvant compound covalently bound to an oxidized carbohydrate residue present at a pre-existing N-linked or O-linked glycan in said glycoprotein. In one embodiment, the N-linked or O-linked glycans are present at serine or threonine residues in said glycoprotein. In another embodiment, the bound immune adjuvant compound does not alter the structure of said glycoprotein. In another embodiment, said bound glycoprotein retains some or all of its natural biological activity.

Another aspect of the invention provides for the types of glycoproteins to which the immune adjuvant compound binds. In one embodiment, said glycoprotein is a natural or synthetic polypeptide. In another embodiment, said glycoprotein is part of a viral-like particle (VLP), a whole virus, or a whole cell. Vaccine compositions comprising the modified glycoproteins of the invention are also included in the invention, for example, compositions comprising one or more isolated modified glycoproteins or peptides, VLPs, whole viruses or whole cells, alone or in combination with known pharmaceutically acceptable excipients and/or adjuvants.

In one embodiment of the invention, the isolated antigen elicits an immune response when administered to a subject. In a further embodiment, the isolated antigen elicits an immune response to an infectious agent or a tumor.

### DETAILED DESCRIPTION OF THE FIGURES

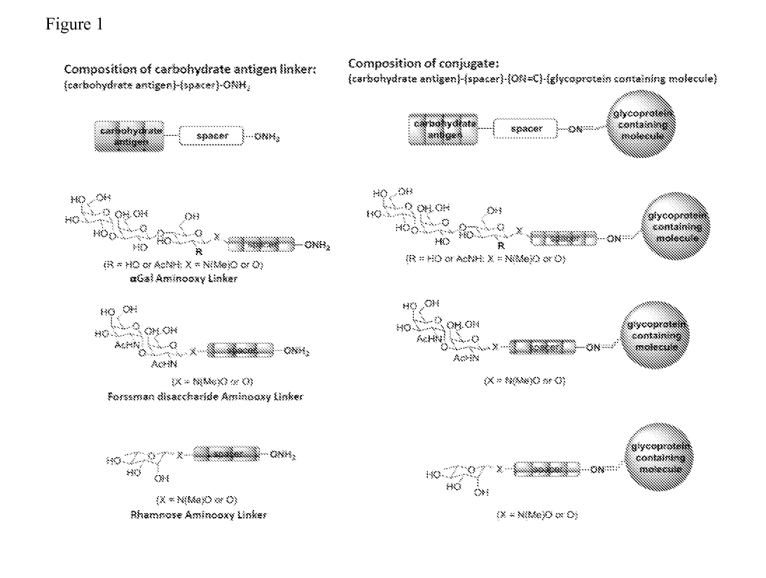

FIG. 1 is a schematic representation of the glycoprotein-carbohydrate epitope conjugate compositions of the invention. The left side of the figure shows the carbohydrate antigen composition comprising an αGal, Forssman disaccharide, or Rhamnose aminooxy linker. The right side of the figure shows these carbohydrate antigen compositions bound through an oxime bond to a glycoprotein antigen.

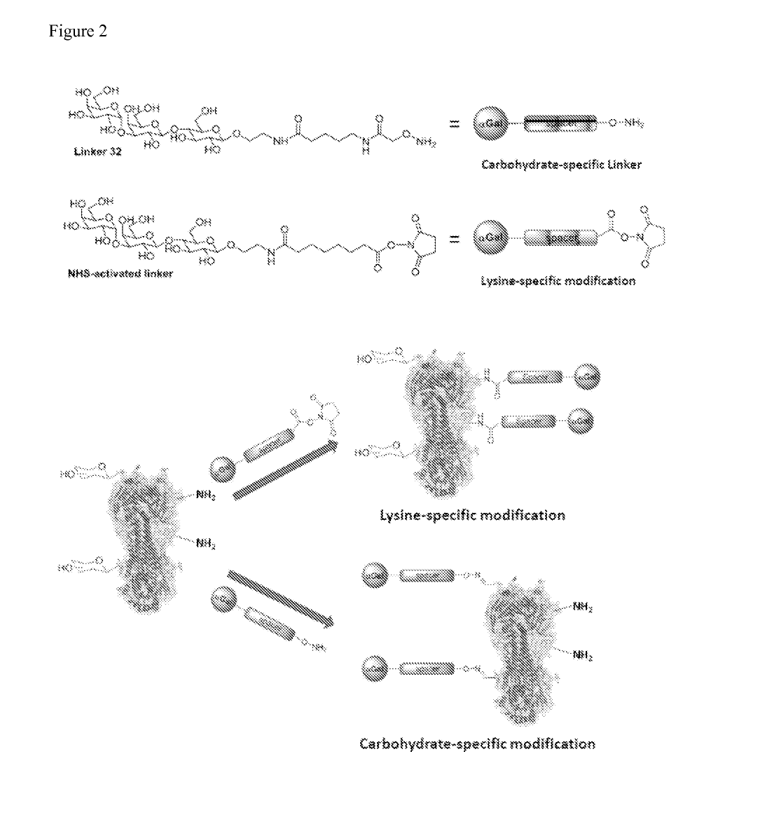

FIG. 2 shows a representation of the differences between the compositions of the invention where the carbohydrate epitope is bound to the glycoprotein antigen at pre-existing carbohydrate residues present on the glycoprotein, and previously described compositions where the carbohydrate epitope is bound to Lysines on the glycoprotein antigen.

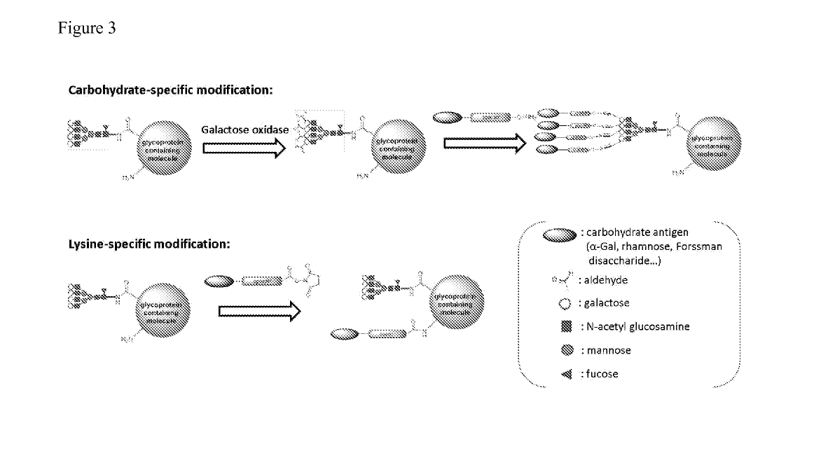

FIG. 3 shows another representation of the differences between the compositions of the invention where the carbohydrate epitope is bound to the glycoprotein antigen at pre-existing carbohydrate residues present on the glycoprotein, and previously described compositions where the carbohydrate epitope is bound to Lysines on the glycoprotein antigen.

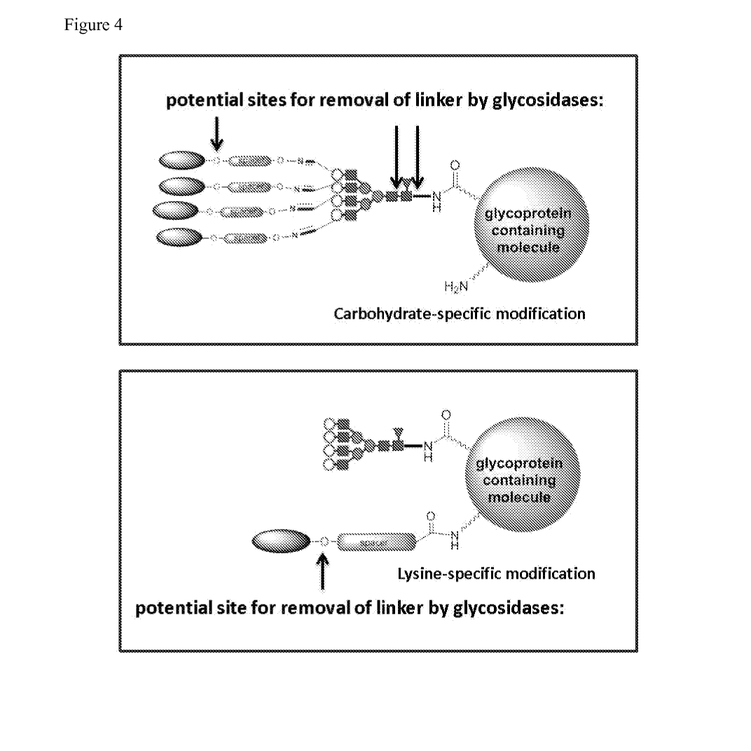

FIG. 4 shows the potential sites for removal of the carbohydrate epitope and linker in carbohydrate specific modified antigen, and lysine-specific modified antigens.

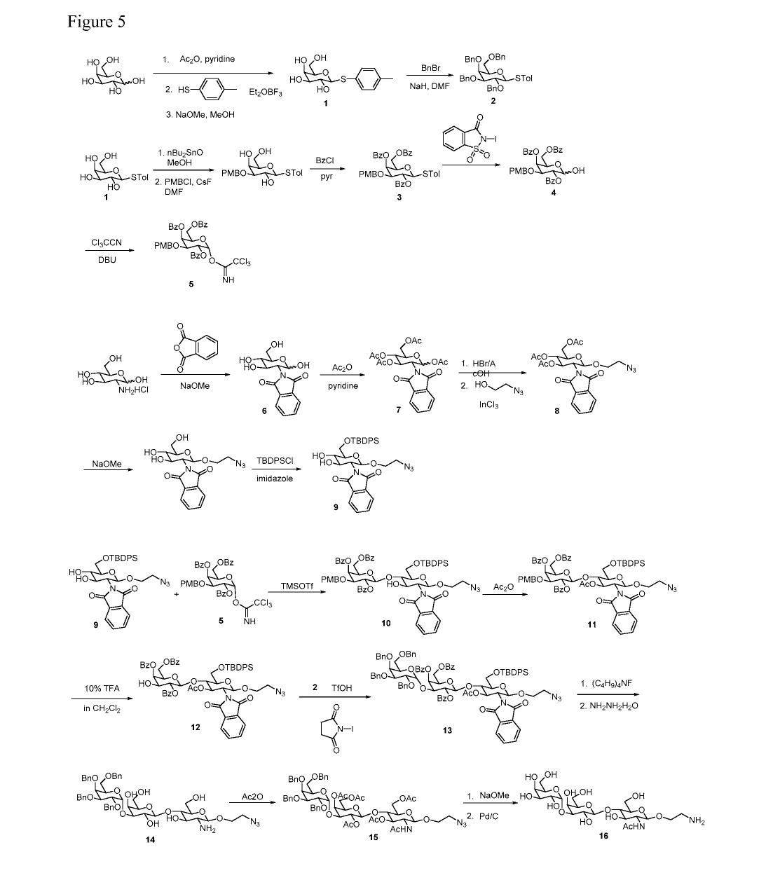

FIG. 5 is a schematic description of synthesis of αGal (GlcNAc containing epitope) amino linkers. See Example 1 for details.

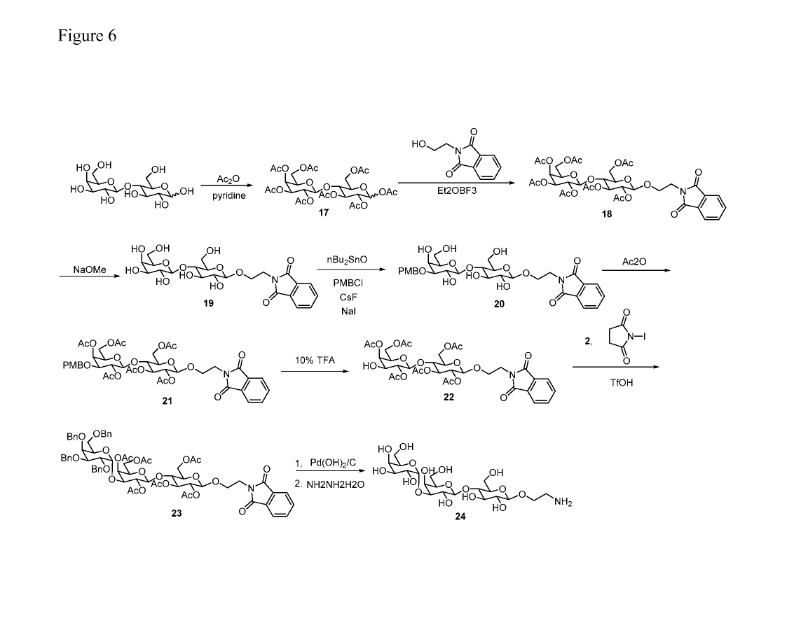

FIG. 6 is a schematic description of synthesis of αGal (Glc containing epitope) amino linkers. See Example 2 for details.

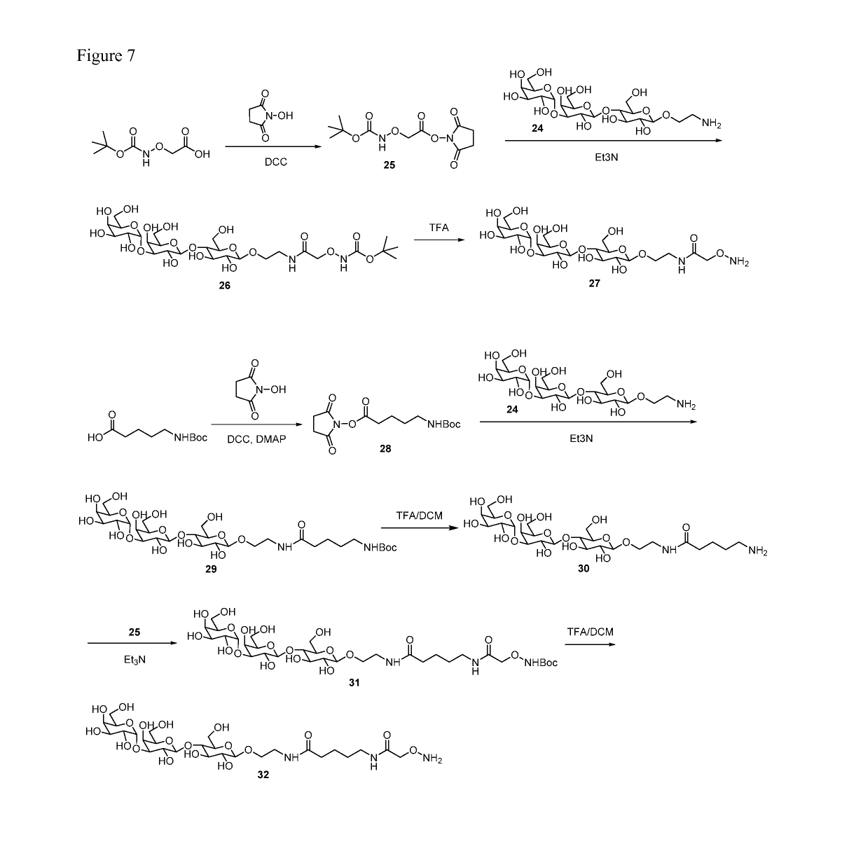

FIG. 7 is a schematic description of synthesis of αGal (Glc containing epitope) aminooxy linkers. See Example 3 for details.

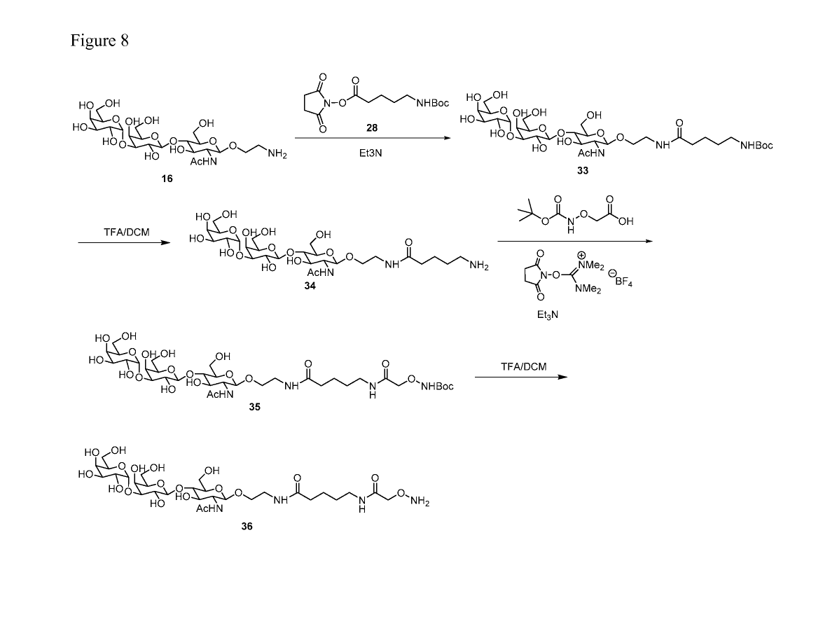

FIG. 8 is a schematic description of synthesis of αGal (GlcNAc containing epitope) aminooxy linkers. See Example 4 for details

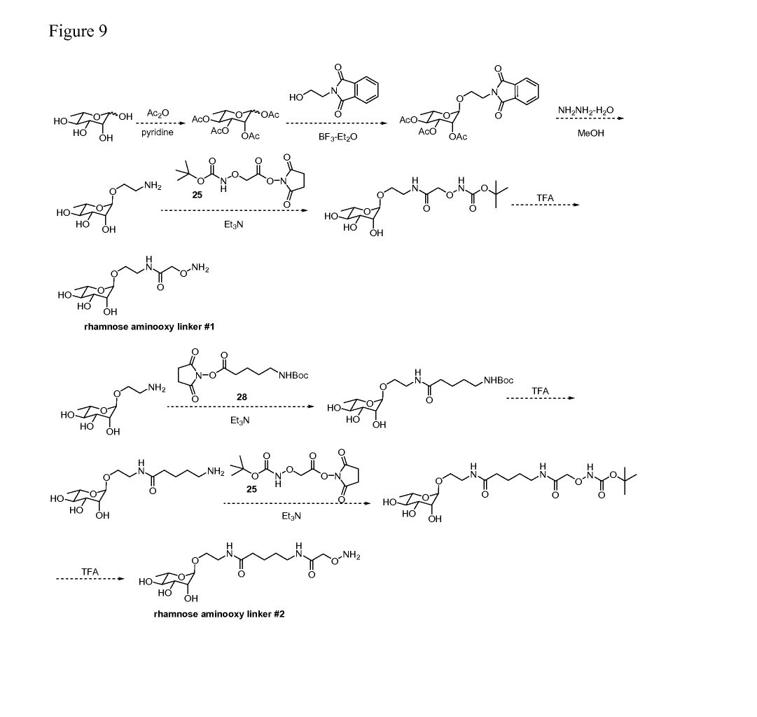

FIG. 9 is a schematic description of synthesis of Rhamnose aminooxy linkers. See Example 5 for details.

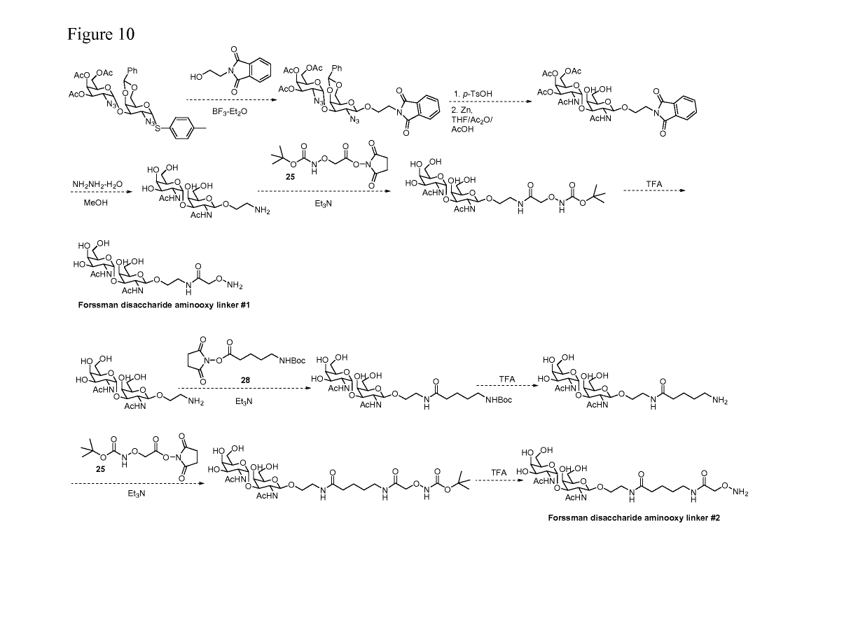

FIG. 10 is a schematic description of synthesis of Forssman disaccharide aminooxy linkers. See Example 6 for details.

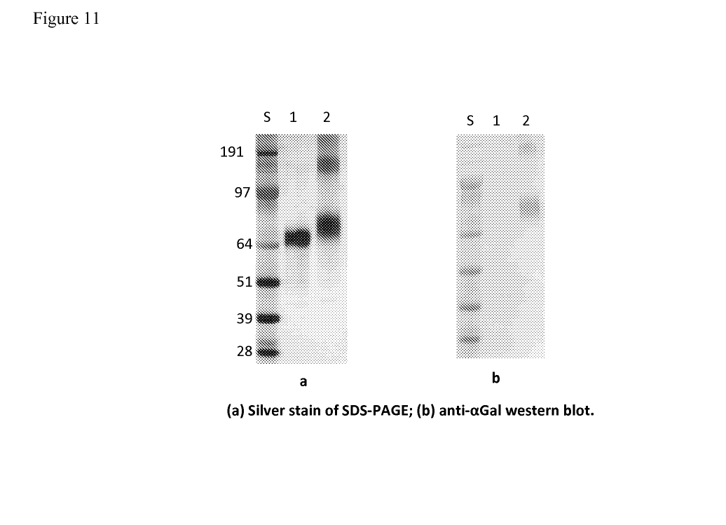

FIG. 11 shows the silver staining of an SDS-PAGE (A) and a Western blot with anti-αGal antibodies (B) of rHA before and after modification with the αGal aminooxy linker 27 (CAL-a08). Lane 1 contains the original rHA, and lane 2 contains oxidized rHA conjugated with CAL-a08. Lane 2 shows distinct migration which indicates that conjugation has occurred. This is confirmed by the Western Blot which shows binding with chicken polyclonal anti-αGal antibodies in lane 2, indicating that the modification had occurred.

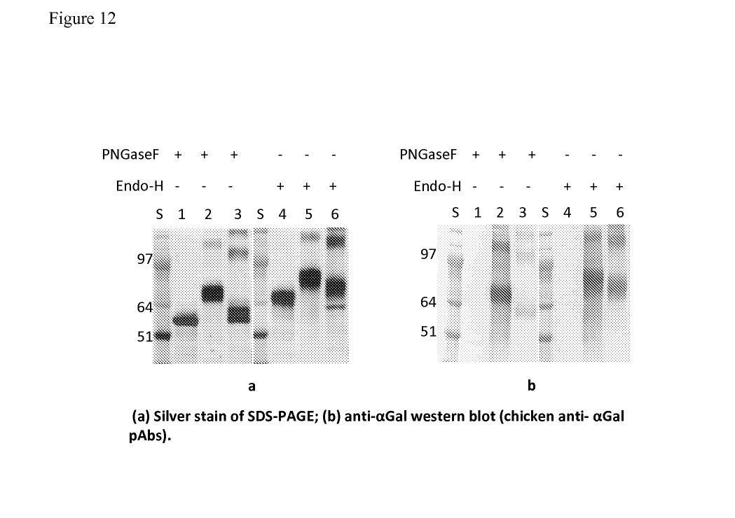

FIG. 12 shows the biological difference between two αGal linker modification technologies: lysine-specific modification and carbohydrate-specific modification after treatment with PNGase and EndoH glycosidases. Panels show the SDS-PAGE (A) and anti-αGal Western Blot (B) for rHA (lanes 1 and 4), rHA modified on the lysine residues with an αGal linker (lanes 2 and 5) and rHA modified on the carbohydrate residues with an αGal linker of the present invention after treatment with the glycosidase PNGaseF (lanes 1 to 3) or and EndoH, respectively (lanes 4 to 6).

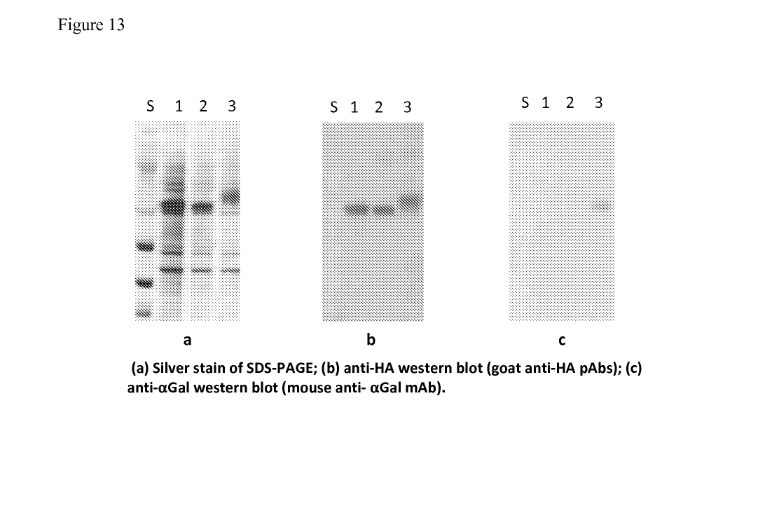

FIG. 13 shows (A) Silver stain of SDS-PAGE, (B) anti-HA western blot, and (C) anti-αGal western blot of a αGal-VLP conjugate. Lane 1 contains the original VLP sample, lane 2 contains the VLP oxidized by GO only, and lane 3 contains the product after conjugation with the αGal aminooxy linker.

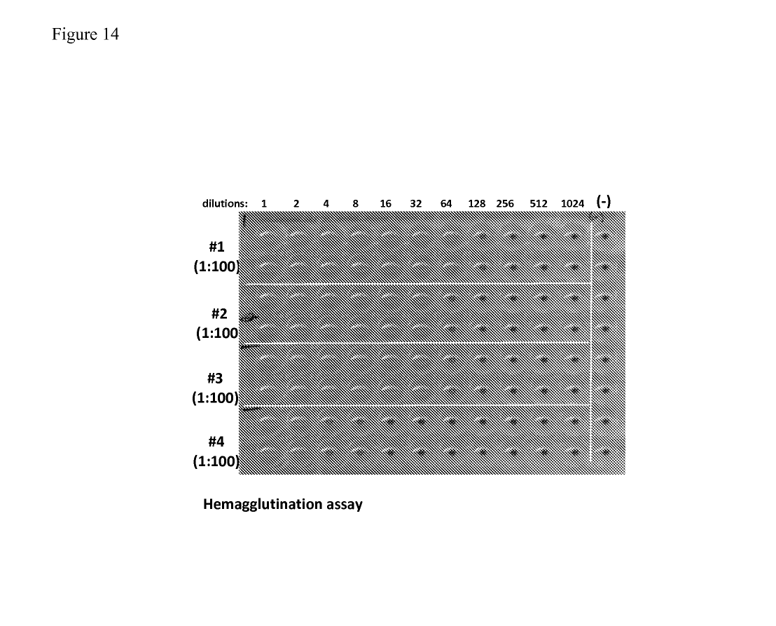

FIG. 14 shows a hemagglutination assay of an αGal-VLP conjugate. The unmodified VLP (Group #1; rows 1&2) induce hemagglutination down to a 1:64 dilution. Oxidized VLPs (Group #2; rows 3&4) and aminooxy linker modified VLPs (group #3; rows 5&6) have similar HA activity at a dilution of 1:32, indicating minimal loss of structure. However, VLPs modified using typical N-hydroxysuccinimide chemistry (Group #4; rows 7&8) lost a significant amount of activity, and were able to induce hemagglutination at only a 1:2 dilution.

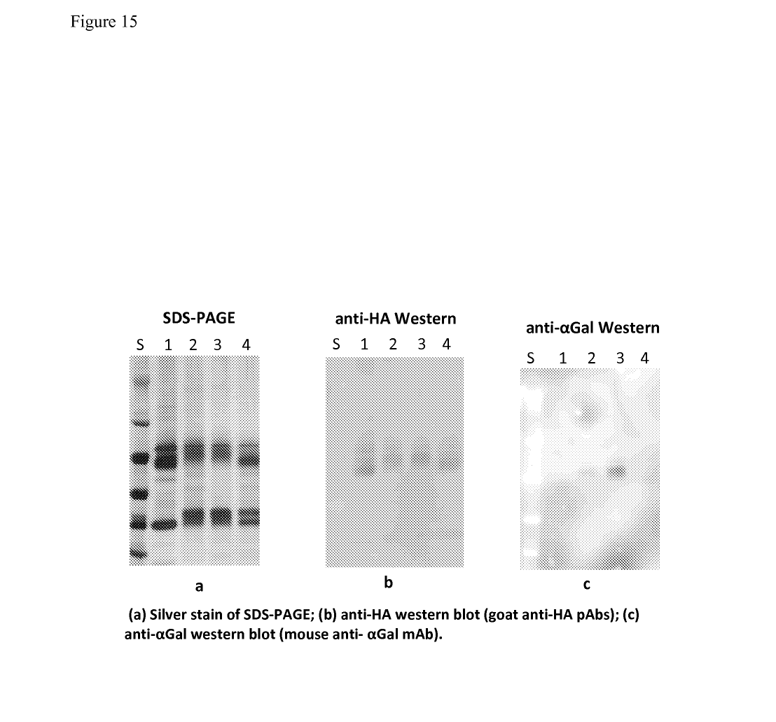

FIG. 15 shows the (A) SDS-PAGE, (B) anti-HA western blot, and (C) anti-αGal western blot for an αGal-Virus conjugate. Lane 1 contains the unmodified virus sample, lanes 2 and 3 contain the αGal aminooxy linker modified inactivated virus, and lane 4 contains the inactivated virus oxidized by GO only. The migration patterns of lanes 2 and 3, and the binding of the anti-αGal antibody to the contents of these lanes indicate that the αGal epitope has been successfully added to the virus.

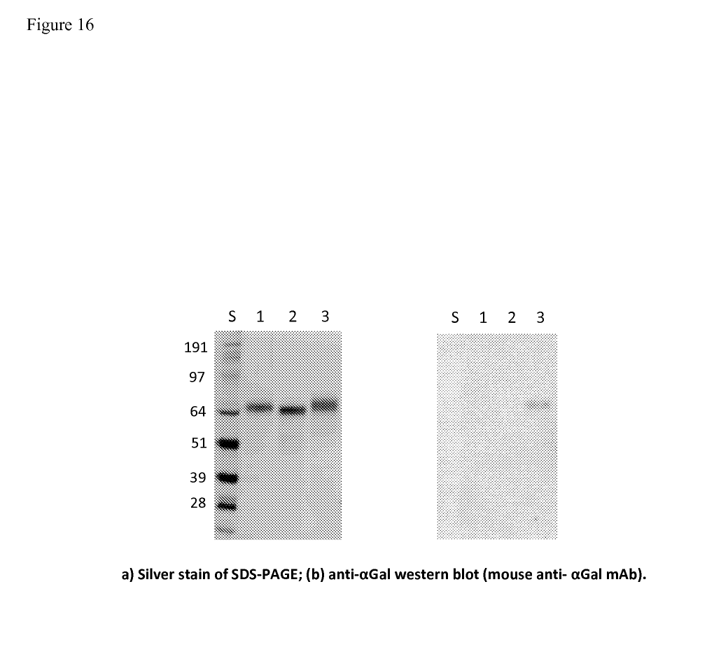

FIG. 16 shows the (A) SDS-PAGE and (B) anti-αGal Western blot for the αGal aminooxy linker 32 (CAL-a11) conjugated to rHA1. Lane 1 contains the unmodified rHA1, lane 2 contains the rHA1 treated with neuraminidase and iGO, and lane 3 contains the αGal-rHA1 conjugate. The migration pattern observed in (A) and the antibody binding observed in (B) indicate successful modification of rHA1 with linker 32.

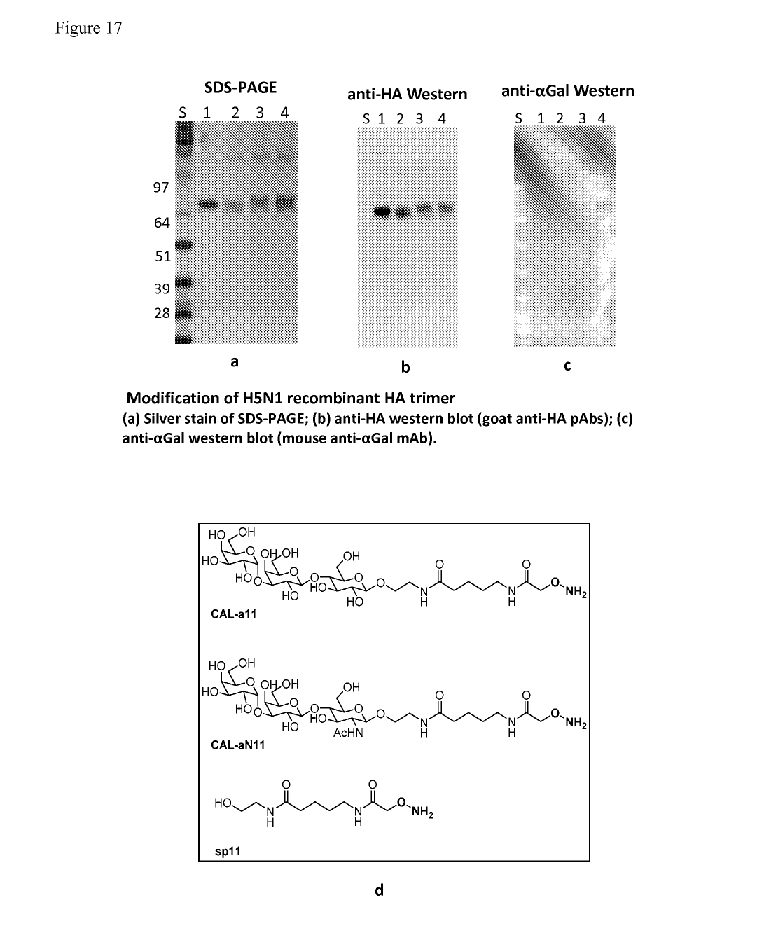

FIG. 17 shows the (A) SDS-PAGE, (B) anti-HA western blot, and (C) anti-αGal western blot for an αGal-H5 conjugate. Lane 1 contains the unmodified H5N1 recombinant HA (H5) sample, lanes 2 contains spacer (sp11) modified H5, and lanes 3 and 4 contain the αGal aminooxy linker CAL-a11 and CAL-aN11 modified H5 respectively. The migration patterns of lanes 3 and 4, and the binding of the anti-αGal antibody to the contents of these lanes indicate that the αGal epitope has been successful added to the H5. (D) Structures of sp11, CAL-a11 and CAL-aN11.

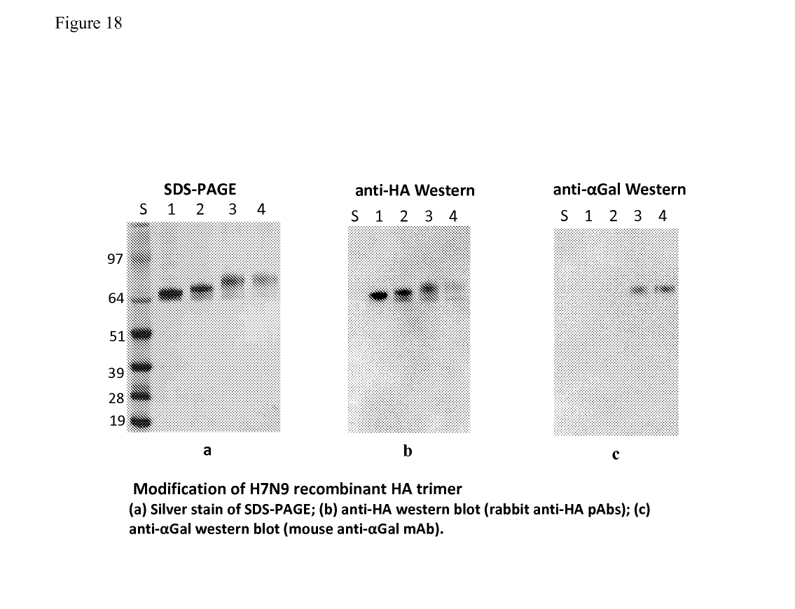

FIG. 18 shows the (A) SDS-PAGE, (B) anti-HA western blot, and (C) anti-αGal western blot for an αGal-H7 conjugate. Lane 1 contains the unmodified H7N9 recombinant HA (H7) sample, lanes 2 contains spacer (sp11) modified H7, and lanes 3 and 4 contain the αGal aminooxy linker CAL-a11 and CAL-aN11 modified H7 respectively. The migration patterns of lanes 2, 3 and 4, and the binding of the anti-αGal antibody to the contents of these lanes indicate that the αGal epitope has been successful added to the H7.

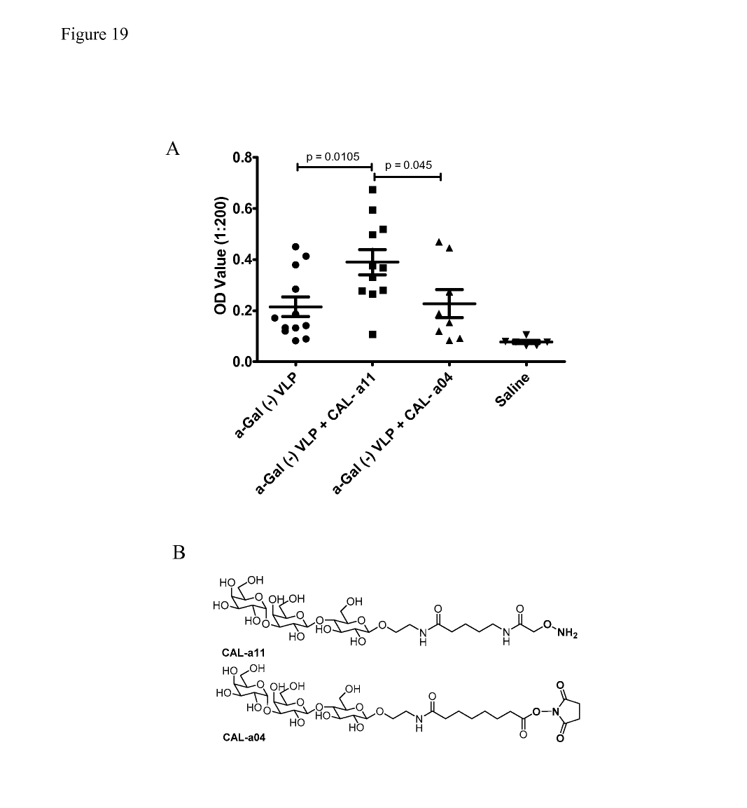

FIG. 19 (A) shows the induction of antibodies against hemagglutinin with αGal linker modified VLPs. The structures of the CAL-a11 (αGal linker for modification of the VLPs at carbohydrate residues) and CAL-a04 linkers (αGal linker for modification of the VLPs at lysine residues) are shown in (B). The OD value reflects the amount of antibody reactivity against recombinant, monomeric HA protein in the sera as measured by ELISA. There is a highly significant difference (p=0.045) in the sera OD values between animals vaccinated with CAL-a11 (VLPs with carbohydrate linker) and CAL-a04 (VLPs with lysine-specific linker). Additionally, CAL-a11 showed a significantly higher OD value than unmodified VLPs alone (p=0.015). There is no statistical difference when comparing mice injected with the unmodified VLPs and those injected with the VLPs modified with the lysine specific linker.

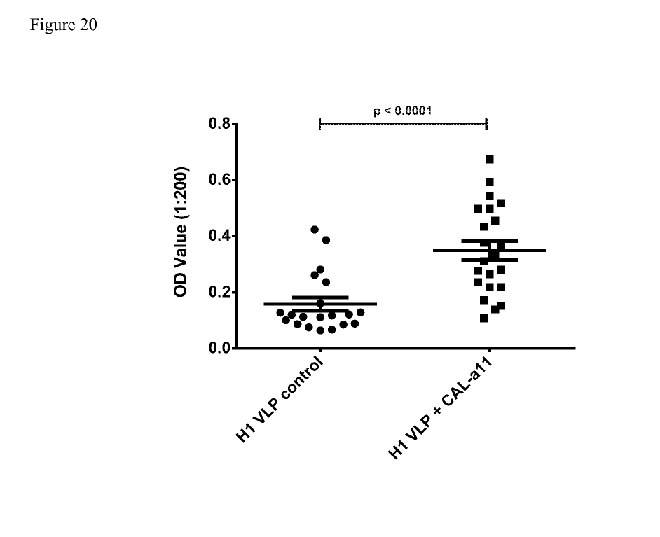

FIG. 20 shows the antibody response after immunization of mice with H1N1 influenza virus-like particles (VLPs) modified with CAL-a11 αGal linker, compared to the antibody responses induced by control VLPs.

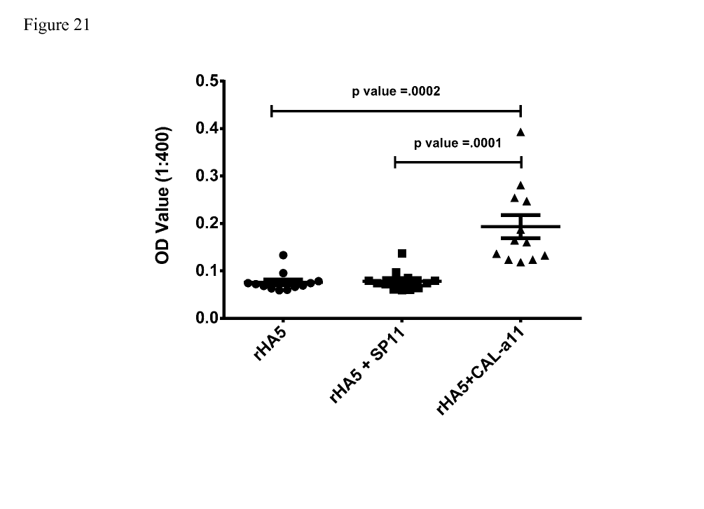

FIG. 21 shows the antibody response after immunization of mice with H5N1 trimeric vaccine modified with CAL-a11 αGal linker, compared to the antibody responses induced by unmodified or spacer only (no αGal-trisaccharide) modified control trimeric H5N1 vaccine.

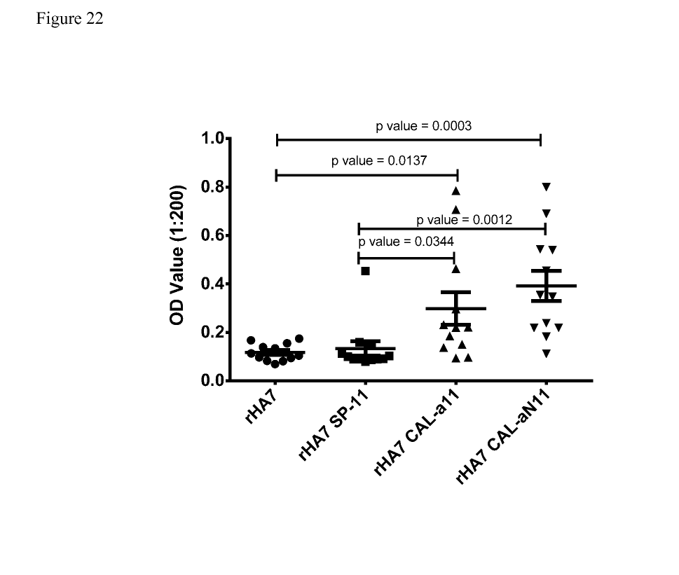

FIG. 22 shows the antibody response after immunization of mice with H7N9 trimeric vaccines. H7N9 trimeric vaccines induce a higher antibody response when modified with CAL-a11 linker and gives an even higher response when the trisaccharide contains a proximal N-acetylglucosamine instead of glucose (CAL-aN11).

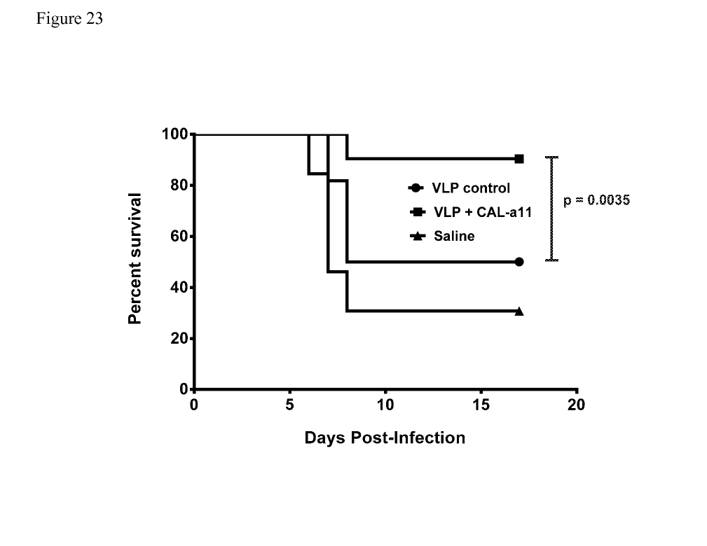

FIG. 23 shows the enhancement in survival and protection after a lethal challenge of mice with H1N1 influenza virus. H1N1 virus-like particles (VLPs) modified with CAL-a11 αGal linker protect mice from influenza mortality.

### DETAILED DESCRIPTION OF THE INVENTION

Various terms relating to the vaccines, compositions and methods of the present invention are used herein above and also throughout the specification and claims.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

The term “αGal epitope” refers to any glycosydic structure composed of at least two monosaccharide units, the first one being a galactosyl or substituted galactosyl residue covalently bond in an α(1-3) bond conformation to a second galactosyl or substituted galactosyl residue, wherein that epitope is recognized by anti-αGal antibodies, including αGal glycomimetic epitopes.

For glycosidic structures, the terms “glycomimetic variant” or “glycomimetic analogs” or “mimotopes” are defined as any glycosidic structure, disaccharide, trisaccharide, tetrasaccharide, pentasaccharide or higher order saccharide structure, branched or linear, substituted or unsubstituted by other chemical groups, that is recognized in an ELISA by antibodies that bind to the reference structure. For example, for the purpose of this definition, the scope of the specificity of anti-αGal antibodies encompasses all antibodies that can be purified by affinity in a column comprising HSA-αGal or BSA-αGal, wherein the αGal epitope bound to HSA or BSA is the Galα1-3Galβ1-4Glc-R trisaccharide plus any linker.

The term “Rhamnose epitope” or “L-Rhamnose epitope” or “L-Rhamnose monosaccharide” refers to the naturally occurring deoxy sugar rhamnose. The Rhamnose epitope which includes Rhamnose glycomimetic epitopes, is recognized by anti- Rhamnose antibodies, and can be bound to a glycosylation site present on a glycoprotein.

The term “Forssman epitope” or “Forssman disaccharide” refers to the Forssman antigen, which is formed by the addition of GalNAc in alpha1-3 linkage to the terminal GalNAc residue of glycoside. The Forssman epitope, which includes Forssman glycomimetic epitopes, is recognized by anti-Forssman antibodies, and can be bound to a glycosylation site present on a glycoprotein.

The term “carbohydrate immune adjuvant” or “carbohydrate epitope” or “carbohydrate antigen” refers to any glycosidic structure, disaccharide, trisaccharide, tetrasaccharide, pentasaccharide or higher order saccharide structure, branched or linear, substituted or unsubstituted by other chemical groups, that can be covalently bound to glycosylation sites present on a glycoprotein antigen, wherein the composition of the carbohydrate epitope and the glycoprotein elicits an immune response when administered to a host.

The term “alkyl” as used herein, means a straight or branched chain hydrocarbon containing from 1 to 30 carbon atoms. As used herein, a substituted alkyl refers to molecules in which carbon atoms in the alkyl chain have been replaced by O, N or S and one or more hydrogen groups have been replaced by hydroxyl, alkyl, amino, carbonyl or sulphydryil. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. Representative examples of a substituted alkyl R₁ according to this definition are: —(CH₂)ₙ—NHC(O)—(CH₂)ₙ—; —(CH₂)ₙ—NHC(O)—(CH₂)ₙ—NHC(O)—(CH₂)ₙ—; —(CH₂)ₙ—OC(O)—(CH₂)ₙ—; —(CH₂)ₙ—(O)CO—(CH₂)ₙ—; —(CH₂)ₙ—C(O)NH—(CH₂)ₙ—NHC(O)—(CH₂)ₙ—; —(CH₂)ₙ—C(O)NH—(CH₂)ₙ—C(O)NH—(CH₂)ₙ—; —(CH₂)ₙ—C(O)—(CH₂)ₙ—O—(CH₂)ₙ—; —(CH₂)ₙ—O—(CH₂)ₙ—O—(CH₂)ₙ—; —(CH₂)ₙ—NHC(O)NH—(CH₂)ₙ—; —(CH₂)ₙ—NHC(O)NH—(CH₂)ₙ—NHC(O)—(CH₂)ₙ—; —(CH₂)ₙ—NHC(O)—(CH₂)ₙ—C(O)NH—(CH₂)ₙ—; —(CH₂)ₙ—(O—(CH₂)ₙ)ₘ—; wherein n and m are 1 to 5.

The term “animal” as used herein should be construed to include all anti-αGal synthesizing animals including those which are not yet known to synthesize anti-αGal. For example, some animals such as those of the avian species, are known not to synthesize αGal epitopes. Due to the unique reciprocal relationship among animals which synthesize either anti-αGal or αGal epitopes, it is believed that many animals heretofore untested in which αGal epitopes are absent may prove to be anti-αGal synthesizing animals. The invention also encompasses these animals.

The term “antibody” includes reference to antigen binding forms of antibodies (e.g., Fab, F(ab)2). The term “antibody” frequently refers to a polypeptide substantially encoded by an immunoglobulin gene or immunoglobulin genes, or fragments thereof which specifically bind and recognize an analyte (antigen). However, while various antibody fragments can be defined in terms of the digestion of an intact antibody, one of skill will appreciate that such fragments may be synthesized de novo either chemically or by utilizing recombinant DNA methodology. Thus, the term antibody, as used herein, also includes antibody fragments such as single chain Fv, chimeric antibodies (i.e., comprising constant and variable regions from different species), humanized antibodies (i.e., comprising a complementarity determining region (CDR) from a non-human source) and heteroconjugate antibodies (e.g., bispecific antibodies).

The term “anti-Forssman” includes any type or subtype of immunoglobulin recognizing a Forssman epitope and/or their glycomimetic variants, of any subtype such as IgG, IgA, IgE or IgM anti-Forssman antibody. For the purpose of this definition, the scope of the specificity of anti-Forssman antibodies encompasses all antibodies that can be purified by affinity in a chromatography column comprising HSA-Forssman or BSA-Forssman, wherein the Rhamnose epitope bound to HSA or BSA is the Forssman disaccharide.

The term “anti-αGal” includes any type or subtype of immunoglobulin recognizing an αGal epitope and/or their glycomimetic variants, of any subtype such as IgG, IgA, IgE or IgM anti-αGal antibody. For the purpose of this definition, the scope of the specificity of anti-αGal antibodies encompasses all antibodies that can be purified by affinity in a chromatography column comprising HSA-αGal or BSA-αGal, wherein the αGal epitope bound to HSA or BSA is the Galα1-3Galβ1-4Glc-R trisaccharide.

The term “anti-Rhamnose” includes any type or subtype of immunoglobulin recognizing a Rhamnose epitope and/or their glycomimetic variants, of any subtype such as IgG, IgA, IgE or IgM anti-Rhamnose antibody. For the purpose of this definition, the scope of the specificity of anti-Rhamnose antibodies encompasses all antibodies that can be purified by affinity in a chromatography column comprising HAS-Rhamnose or BSA-Rhamnose, wherein the Rhamnose epitope bound to HSA or BSA is the Rhamnose monosaccharide.

As used herein, the term “antigen” is meant any biological molecule (proteins, peptides, lipoproteins, glycans, glycoproteins) that is capable of eliciting an immune response against itself or portions thereof, including but not limited to, polypeptides, viral-like particles (VLPs), tumor associated antigens and viral, bacterial, parasitic and fungal antigens.

As used herein, the term “antigen presentation” refers to the biological mechanism by which macrophages, dendritic cells, B cells and other types of antigen presenting cells process internal or external antigens into subfragments of those molecules and present them complexed with class I or class II major histocompatibility complex or CD1 molecules on the surface of the cell. This process leads to growth stimulation of other types of cells of the immune system (such as CD4+, CD8+, B and NK cells), which are able to specifically recognize those complexes and mediate an immune response against those antigens or cells displaying those antigens.

The term “chemical” with reference to the addition of an epitope shall mean that addition of an epitope in that does not occur within an intact, live cell.

The terms “MHC” (Major Histocompatibility Complex) or “HLA” (Human Leukocyte Antigen) refer to the histocompatibility antigens of mouse and human, respectively. Herein, MHC of HLA are used indistinctly to refer to the histocompatibility antigens, without a species restriction, and teachings referring to MHC also apply to HLA and vice versa.

With respect to proteins or peptides, the term “isolated protein (or peptide)” or “isolated and purified protein (or peptide)” or “isolated TAA protein” is sometimes used herein. This term may refer to a protein that has been sufficiently separated from other proteins with which it would naturally be associated, so as to exist in “substantially pure” form. Alternatively, this term may refer to a protein produced by expression of an isolated nucleic acid molecule.

As used herein, “mimotope” refers to molecular variants of certain epitopes that can mimic the immunologic properties of said epitopes in terms of its binding to the same antibodies or being recognized by the same MHC molecules or T cell receptors.

The term “opsonization” of an antigen or a tumor cell may be used to refer to binding of the epitopes present in the antigen or on the surface of a tumor cell by antibodies thereby forming immunocomplexes and enhancing phagocytosis of the opsonized antigen or tumor cell by macrophages, dendritic cells, B cells or other types of antigen presenting cells through binding of the Fc portion of the antibodies to the FcγR receptors present on the surface of antigen presenting cells.

The term “peptide” refers to a polymer of about 2-50 amino acids or any length in between. Peptides can be derived from proteolytic cleavage of a larger precursor protein by proteases, or can be chemically synthesized using methods of solid phase synthesis. Synthetic peptides can comprise non-natural amino acids, such as homoserine or homocysteine to serve as substrates to introduce further chemical modifications such as chemical linkers or sugar moieties. In addition, synthetic peptides can include derivatized glyco-aminoacids to serve as precursors of glycopeptides containing the carbohydrate epitope or its glycomimetic variants.

The terms “protein” or “polypeptide” are used interchangeably herein to refer to a polymer of amino acid residues larger than about 50 amino acids. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, the protein is specifically reactive to antibodies elicited to the same protein but consisting entirely of naturally occurring amino acids. The terms “polypeptide” and “protein” are also inclusive of modifications including, but not limited to, phosphorylation, glycosylation, lipid attachment, sulfation, gamma carboxylation of glutamic acid residues, hydroxylation and ADP-ribosylation.

As used herein, “glycoprotein antigen” or “glycoprotein containing antigen” refers to a polypeptide, or fragment thereof containing oligosaccharide chains (glycans) that exists as an isolated polypeptide, or is part of a higher order structure including but not limited to, a VLPs, whole virus, or whole cells. The glycoprotein antigen can be a polypeptide produced by a cell, either naturally or recombinantly, or the glycoprotein antigen can be a synthetic polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “therapeutically effective amount” is meant an amount of treatment composition sufficient to elicit a measurable increase in a desired immuno response, which can further result in a decrease in the number, quality or replication rate of previously existing tumor cells or virus-infected cells.

The term “tumor cell” refers to a cell which is a component of a tumor in an animal, or a cell which is determined to be destined to become a component of a tumor, i.e., a cell which is a component of a precancerous lesion in an animal, or a cell line established in vitro from a primary tumor. Included within this definition are malignant cells of the hematopoietic system which do not form solid tumors such as leukemias, lymphomas and myelomas.

The term “tumor” is defined as one or more tumor cells capable of forming an invasive mass that can progressively displace or destroy normal tissues.

The term “malignant tumor” refers to those tumors formed by tumor cells that can develop the property of dissemination beyond their original site of occurrence.

The term “Tumor Associated Antigens” or “TAA” refers to any protein or peptide expressed by tumor cells that is able to elicit an immune response in a subject, either spontaneously or after vaccination. TAAs comprise several classes of antigens: 1) Class I HLA restricted cancer testis antigens which are expressed normally in the testis or in some tumors but not in normal tissues, including but not limited to antigens from the MAGE, BAGE, GAGE, NY-ESO and BORIS families; 2) Class I HLA restricted differentiation antigens, including but not limited to melanocyte differentiation antigens such as MART-1, gp100, PSA, Tyrosinase, TRP-1 and TRP-2; 3) Class I HLA restricted widely expressed antigens, which are antigens expressed both in normal and tumor tissue though at different levels or altered translation products, including but not limited to CEA, HER2/neu, hTERT, MUC1, MUC2 and WT1; 4) Class I HLA restricted tumor specific antigens which are unique antigens that arise from mutations of normal genes including but not limited to β-catenin, α-fetoprotein, MUM, RAGE, SART, etc; 5) Class II HLA restricted antigens, which are antigens from the previous classes that are able to stimulate CD4+ T cell responses, including but not limited to member of the families of melanocyte differentiation antigens such as gp100, MAGE, MART, MUC, NY-ESO, PSA, Tyrosinase; and 6) Fusion proteins, which are proteins created by chromosomal rearrangements such as deletions, translocations, inversions or duplications that result in a new protein expressed exclusively by the tumor cells, such as Bcr-Abl.

The term “TAA-derived peptides” refer to amino acid sequences of 5, 6, 7, 8, 9, 10, 11, 12, 13, 14, 15, 16, 17, 18, 19 or 20 amino acids that bind to MHC (or HLA) class I or class II molecules, and that have at least 70% amino acid identity sequence with an amino acid sequence contained within the corresponding TAA. Peptide sequences which have been optimized for enhanced binding to certain allelic variants of MHC class I or class II are also included within this class of peptides. In one embodiment, the TAA peptides further comprise at least one or more αGal acceptor amino acids and/or an affinity purification tag. In another embodiment, αGal acceptor amino acids flank the TAA peptide.

As used herein, “vaccine” refers to any antigenic composition used to elicit an immune response. The antigenic composition can be unmodified peptides, glycosylated peptides, purified or recombinant proteins or glycoproteins, VLPs, whole viruses or whole cells or cell fractions. A vaccine can be used therapeutically to ameliorate the symptoms of a disease, or prophylactically, to prevent the onset of a disease.

The term “treat” or “treating” with respect to tumor cells refers to stopping the progression of said cells, slowing down growth, inducing regression, or amelioration of symptoms associated with the presence of said cells.

The term “xenogeneic” refers to a cell or protein that derives from a different animal species than the animal species that becomes the recipient animal host in a transplantation or vaccination procedure.

The term “allogeneic” refers to a cell or protein that is of the same animal species but genetically different in one or more genetic loci as the animal that becomes the “recipient host”. This usually applies to cells transplanted from one animal to another non-identical animal of the same species, or to vaccination of an animal with a protein or antigen from a different strain which may contain differences in the amino acid sequence or post-translational modifications.

The term “syngeneic” refers to a cell or protein which is of the same animal species and has the same genetic or amino acid sequence composition for most genotypic and phenotypic markers as the animal who becomes the recipient host of that cell line in a transplantation or vaccination procedure. This usually applies to cells transplanted from identical twins or may be applied to cells transplanted between highly inbred animals.

The present invention provides an immunogenic composition comprising a glycoprotein antigen in association with a carbohydrate epitope, including but not limited to, the αGal, Rhamnose monosaccharide (e.g. L-Rhamnose) and/or the Forssman disaccharide epitopes, and provides methods for inducing an immune response in an animal, and methods of making the immunogenic compositions. Non-limiting examples of glycoprotein antigens include, but are not limited to, isolated glycoproteins, and glycoproteins which are part of a higher order structure such as VLPs, whole viruses, and/or whole cells. The invention takes advantage of the naturally high titers of antibodies to the carbohydrate epitopes in animals to target vaccine compositions to antigen presenting cells for effective processing and presentation to the immune system.

The binding of natural IgG or IgM antibodies to the carbohydrate epitopes present in the modified antigen facilitates the formation of immunocomplexes and triggers complement activation and opsonization of the immunocomplex by C3b and C3d molecules, which can target the immunocomplex to follicular dendritic cells and B cells via CD21 and CD35, thereby augmenting the immune response. FcγR receptor mediated phagocytosis of IgG immunocomplexes by DCs is a very efficient mechanism of antigen uptake and processing. Additionally, complement-activation at the site of vaccination generates a “danger signal” which has numerous implications for the kind of immune response that will be generated (Matzinger 2002; Perez-Diez et al. 2002). Danger signals are recognized as crucial components for APC activation and differentiation to mature DCs. Furthermore, complement activation has chemo-attractant properties that, similarly to GM-CSF, result in inflammation and recruitment of APCs.

Different antigen uptake and processing pathways control the presentation of antigenic peptides by either MHC class I molecules to CD8+ T cells (endogenous pathway) or MHC class II molecules to CD4+ T cells (exogenous pathway). Vaccines that are composed of exogenous antigens use mainly the exogenous pathway for the delivery of antigen to APCs. This, in turn, favors the stimulation of CD4+ T cells and the production of antibodies. To deliver exogenous antigens to the endogenous pathway in order to elicit a cellular mediated response, the engagement of the FcγR receptor to mediate antigen uptake of immunocomplexes is very important as it stimulates the cross-presentation pathway (Heath and Carbone 2001). Studies indicate that, in addition to classical CD4+ priming, antigen acquired through endocytosis by DC through FcγR results in the induction of T cell effector immunity resulting in T_{H}1 and class I restricted CD8+ T cell priming. Furthermore, engagement of FcγR also induces DC activation and maturation. Thus, the existing evidence indicates that antigenic targeting to FcγR on DC accomplishes several important aspects of T cell priming important for induction of an immune response: facilitated uptake of antigen, class I and class II antigen presentation and induction of DC activation and maturation.

The compositions of the invention described herein are constructed following a modification strategy that specifically targets carbohydrate epitopes to the carbohydrate residues on glycoprotein antigens. The compositions resulting from this method retain their original biological activities since the glycoprotein's backbone is intact throughout the entire modification process, thereby retaining its native conformation. The invention selectively introduces carbohydrate epitopes to carbohydrate residues on a glycoprotein using a combination of NaIO₄, galactose oxidase (GO) or its derivatives, and an aminooxy linker.

The carbohydrate epitopes of the present invention can be connected to the glycoprotein antigen through various linkers comprising any linear or branched alkyl group of 1 to 30 carbon atoms, wherein one or more carbon atoms in such alkyl group can be substituted by O, S, or N and wherein one or more hydrogens can be substituted by hydroxyl, carbonyl, alkyl, sulphydryl or amino groups. Examples of various linkers can be found, for example, in U.S. Pat. No. 8,357,777 which is hereby incorporated by reference in its entirety. In one embodiment, the linker is a natural structure that is susceptible to metabolism and/or cleaving in the cell. In another embodiment, the linker is soluble. In one embodiment, the carbohydrate epitope is connected to the linker through a N(Me)O group. In one embodiment, the carbohydrate epitope is connected to the linker through an Oxygen.

This strategy targets surface existing carbohydrate moieties, and not amino acid residues which are affected by other common means of modifying polypeptides (e.g. lysine modification by NHS or cysteine modification by Maleimide). The new carbohydrate linkers will attach to pre-existing N-glycans or O-glycans on the glycoprotein antigen, and can therefore be removed by natural N-glycosidases and O-glycosidases that typically play a role during antigen processing and presentation. The method described herein does not block the original antigenic regions present on the glycoprotein or change the biological activity of the glycoprotein after modifications.

The carbohydrate epitope and linker are attached to the oxidized glycosylation sites present on the glycoprotein through an aminoxy group at the end of the linker (FIG. 1). This aminoxy group, when reacted with the aldehyde in the oxidized glycosylation sites will form an oxime bond with the carbohydrate residue on the glycoprotein antigen to generate a modified glycoprotein of structure Su-O—R₁—O—N═CR, where CR represents the carbohydrate and amino acid residue, or glycosylated amino acid residue, of said glycoprotein.

There are several advantages to the association of the carbohydrate epitope with glycosylation sites present on the glycoprotein antigen through natural, hydrolyzable bonds. First, the bonds formed are reversible natural bonds which can be hydrolyzed by naturally produced enzymes. Upon entry into the cell, these bonds can be cleaved by enzymes already present, thereby releasing the carbohydrate antigen from the complex. Second, there are more potential cleavage sites whereby the carbohydrate epitopes can be removed from the glycoprotein antigen (See, FIGS. 3 & 4). This can result in the entire carbohydrate epitope being removed from the glycoprotein antigen, leaving only the protein antigen to be cleaved by proteases into smaller peptides that can be presented by the APCs in the context of both MHC (or HLA) class I or II, thereby inducing a robust immune response against the glycoprotein antigen.

The compositions of the invention are made through a chemical process whereby the composition is produced by reacting one or more carbohydrate residues present on the glycoprotein antigen with a carbohydrate epitope and linker, to selectively attach the carbohydrate epitope to an oxidized carbohydrate residues present on the glycoprotein. Briefly, the carbohydrate residues on the glycoprotein antigen are oxidized to produce a reactive carbonyl group which is then reacted with the aminooxy group on the carbohydrate epitope comprising a linker to form an oxime bond. The oxidizing enzyme may be free or immobilized.

The oxidizing step is performed using NaIO₄, Galactose oxidase (GO), or an engineered variant of GO, depending upon the glycoprotein antigen being modified. NaIO₄ is not suitable for all targets since it has no selectivity, other than differentiating sialic acid and other carbohydrates during oxidations. Additionally, NaIO₄ might destroy the higher order structure of a complex glycoprotein antigen due to unpredictable side reactions. Galactose oxidase provides a much specific and milder reaction condition and has exclusive selectivity towards terminal galactose and N-acetylgalactosamine. Purified glycoproteins that are not part of a higher order structure can be oxidized by NaIO₄ to attach the carbohydrate linkers described herein. Galactose oxidase (GO) and its variants can be used to modify glycoproteins with terminal galactose, N-acetylgalactosamine, or sialic acid, or glycoproteins that are part of a higher order structure. Known variants of galactose oxidase include, for example, those described in U.S. Pat. No. 6,498,026 which is hereby incorporated by reference in its entirety. This method produces modified molecules similar to those obtained by enzymatic or biological modifications.

In some embodiments, NaIO₄ is used to oxidize the carbohydrate residues present on a purified, isolated glycoprotein. In certain embodiments, GO or an engineered variant thereof, is used to oxidize the carbohydrate residues present on a glycoprotein antigen that is part of a higher order structure. In other embodiments, an engineered GO is used to oxidize the carbohydrate residues on a glycoprotein which lacks a terminal galactose, N-acetylgalactosamine, or sialic acid. In other embodiments, the GO or engineered variant thereof is immobilized. In yet another embodiment, the GO or engineered variant thereof is free.

As described herein, the carbohydrate epitope and linker are attached through a covalent bond to the glycoprotein antigen at one or more oxidized carbohydrate residues present on the glycoprotein. In some embodiments, the carbohydrate epitope and linker are bound to oxidized carbohydrate residues present at one or more pre-existing N-linked or O-linked glycans in the glycoprotein. In one embodiment, the carbohydrate residue is a galactose residue. In another embodiment, the oxidation of the carbohydrate residue present at pre-existing N-linked or O-linked glycans in the glycoprotein is performed with galactose oxidase.

Carbohydrate epitopes with the generic structure Su-O—R₁—ONH₂ are synthesized by the methods of the present invention. Su can be a monosaccharide, disaccharide, trisaccharide, tetrasaccharide, or pentasaccharide, and R₁ is a linker comprising any linear or branched alkyl group of 1 to 30 carbon atoms, wherein one or more carbon atoms in such alkyl group can be substituted by O, S, or N and wherein one or more hydrogens can be substituted by hydroxyl, carbonyl, alkyl, sulphydryl or amino groups. In one embodiment, such atom substitutions create one or more ester, ether, thio, amide or carbamate groups situated at any position within the R₁ alkyl chain. The molecules of the present invention covalently join the Su moiety to the R1 linker via a —O-glycosidic bond, which is an advantage over more common synthetic bonds of the structure —N(CH₃)—O—, which are not susceptible to hydrolysis by O-glycosydases. The resulting molecule is then reacted with the carbonyl groups on an oxidized glycoprotein antigen, and an oxime bond is formed between the carbonyl group on the glycoprotein and the aminooxy group on the carbohydrate antigen to generate a modified glycoprotein of structure Su-O—R₁—O—N═CR, where CR represents the carbohydrate and amino acid residue, or glycosylated amino acid residue, of said glycoprotein. The methods and compositions described herein for the synthesis of αGal-O—R₁—ONH₂ activated molecules apply to any saccharide, including, but not limited to monosaccharides, disaccharides, trisaccharides, tetrasaccharides and/or pentasaccharides to which humans have high levels of pre-existing antibodies, for example αGal and derivatives thereof.

The present invention provides methods for the addition of different carbohydrate epitopes to glycoprotein antigens to increase the antigen's immunogenicity. The presence of the carbohydrate epitope attached to the glycoprotein antigen promotes the in vivo formation of immunocomplexes with natural antibodies to the carbohydrate epitope. The binding of natural IgG or IgM antibodies to the carbohydrate epitopes facilitates the formation of immunocomplexes which triggers complement activation and opsonization of the immunocomplex by C3b and C3d molecules, which can target the immunocomplex to follicular dendritic cells and B cells via CD21 and CD35, thereby augmenting the immune response.

The carbohydrate epitope can be any saccharide, including but not limited to monosaccharides, disaccharides, trisaccharides, tetrasaccharides, or pentasaccharides to which humans have high levels of pre-existing antibodies. The glycoprotein antigens described herein may be bound to one or more carbohydrate epitopes, optionally through a chemical linker. These carbohydrate epitopes that can be covalently bound to the glycoprotein antigen include, but are not limited to, the αGal, L-Rhamnose, and Forssman epitopes and variants thereof. In one embodiment, the carbohydrate epitope is αGal or a variant thereof. In another embodiment, the carbohydrate epitope is L-Rhamnose or a variant thereof. In another embodiment, the carbohydrate epitope is the Forssman epitope or variant thereof.

Natural anti-αGal antibodies are of polyclonal nature and synthesized by 1% of circulating B cells. They are present in serum and human secretions and represented by IgM, IgG and IgA classes. The main epitope recognized by these antibodies is the αGal epitope (Galα1-3Galβ1-4NAcGlc-R) but they can also recognize other carbohydrates of similar structures such as Galα1-3Galβ1-4Glc-R, Galα1-3Galβ1-4NAcGlcβ1-3Galβ1-4Glcβ-R, Galα1-3Glc (melibiose), α-methyl galactoside, Galα1-6Galα1-6Glcβ (1-2)Fru (stachyose), Galα1-3(Fucα1-2)Gal-R (Blood B type epitope), Galα1-3Gal and Galα1-3Gal-R (Galili et al. 1987; Galili et al. 1985; Galili et al. 1984). Similarly, non-natural synthetic analogs of the αGal epitope have been described to bind anti-αGal antibodies and their use has been proposed to deplete natural anti-αGal antibodies from human sera in order to prevent rejection of xenogeneic transplants (Janczuk et al. 2002; Wang et al. 1999). Therefore, glycomimetic analogs of the αGal epitope could also be used to promote the in vivo formation of immunocomplexes for vaccination purposes.

Similarly, natural antibodies against Forssman antigen and Rhamnose carbohydrate are present in very high levels in human plasma (REF) and therefore constitute a preferred candidate for the formation of in vivo immunocomplexes with antigens bearing these carbohydrates.

Theoretically, there is no limitation for the identity or properties of the antigen used for vaccination. The compositions and methods may employ any glycoprotein antigen in association with a carbohydrate epitope. Generally, the composition will comprise a glycoprotein antigen that can be oxidized at one or more glycosylation sites to form carbonyl groups on the surface of the protein and can include any natural or synthetic glycoprotein existing by itself, or as part of a higher order structure such as a VLP, whole virus, or whole cell.

In certain embodiments, the glycoprotein antigen is an isolated glycoprotein. Glycoproteins which may be comprised in the isolated antigens of the invention include, but are not limited to, tumor associated antigens (TAAs), isolated coat polypeptides or fragments thereof from viruses, isolated polypeptides or fragments thereof expressed on the surface of cells, autoantigens, synthetic polypeptides or fragments thereof, allergans, tolerogens, and/or immunoglobulin binding proteins (e.g. Protein A, Protein G, and/or Protein L).

In certain embodiments, the glycoprotein antigen is part of a higher order structure. In certain embodiments, the glycoprotein antigen is part of a polypeptide fusion and/or complexes. In another embodiment, the glycoprotein antigen is part of a VLP. In another embodiment, the glycoprotein antigen is part of a whole virus. In another embodiment, the glycoprotein antigen is part of a whole cell.

In certain embodiments, the glycoprotein antigens comprise VLPs. Non-limiting examples of VLPs include, but are not limited to, VLPs derived from the Hepatitis B virus, the Influenza virus (e.g. H5N1), Parvoviridae (e.g. adeno-associated virus), Herpesviridiae (HSV) Papillomaviridiae (HPV), (Retroviridae (e.g. HIV), and/or Flaviviridae (e.g. West Nile Virus).

In certain embodiments, the glycoprotein antigens comprise whole viruses. Non-limiting examples of whole viruses include, but are not limited to, double stranded DNA viruses (e.g. Adenoviruses, Herpesviruses, Poxviruses), single stranded DNA viruses (e.g. Parvoviruses), double stranded RNA viruses (e.g. Reoviruses), single stranded RNA viruses (e.g. Picornaviruses, Togaviruse, Orthomyxoviruses, Rhabdoviruses), single stranded RNA-RT viruses (e.g. Retroviruses) and/or double stranded DNA-RT viruses (e.g. Hepadnaviruses). In a particular embodiment, the whole viruses are Human Immunodeficiency Virus (HIV-1 and HIV-2), influenza, hepatitis B (HBV), hepatitis C (HCV), herpes simplex virus (HSV-1) and human papilloma virus (HPV).

In certain embodiments, the glycoprotein antigen of the invention is one or more whole cells comprising the modified glycoprotein. Non-limiting examples of whole cells include, but are not limited to bacteria, and/or tumor cells. In one embodiment, the cells are attenuated and/or killed.

In one embodiment, the glycoprotein antigen of the invention is one or more bacterial cells comprising the modified glycoprotein. Non-limiting examples of bacterial cells include, but are not limited to, staphlococcus infections, streptococcus infections, mycobacterial infections, bacillus infections, Salmonella infections, Vibrio infections, spirochete infections, and Neisseria infections.

In one embodiment, the glycoprotein antigen of the invention is one or more tumor cells comprising the modified glycoprotein. Non-limiting examples of tumor cells include, but are not limited to, malignant and non-malignant tumors. Cells from malignant (including primary and metastatic) tumors include, but are not limited to, those occurring in the adrenal glands; bladder; bone; breast; cervix; endocrine glands (including thyroid glands, the pituitary gland, and the pancreas); colon; rectum; heart; hematopoietic tissue; kidney; liver; lung; muscle; nervous system; brain; eye; oral cavity; pharynx; larynx; ovaries; penis; prostate; skin (including melanoma); testicles; thymus; and uterus. Examples of such tumors include apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), plasmacytoma, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myoma, myosarcoma, rhabdomyoma, rhabdomyosarcoma, ependymoma, ganglioncuroma, glioma, medulloblastoma, meningioma, neurilemnnoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyorna, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewing's experimental, Kaposi's, and mast-cell), neoplasms and for other such cells.

In one embodiment of the invention, the compositions of the invention elicit an immune response when administered to a subject. In a further embodiment, the isolated antigen elicits an immune response to an infectious agent or a tumor. In a further embodiment, the subject is human.

In one embodiment, the compositions of the invention provide a method for inducing an immune-mediated destruction of tumor cells, virus-infected cells, or bacterial-infected cells in an animal. In another embodiment, the method comprises administering to an animal in thereof, a composition of the invention described herein.

In one embodiment, the animal has cancer or an uncontrolled cellular growth. In a further embodiment, the compositions of the invention comprise tumor cells and/or other glycoprotein antigens derived from tumor cells as the immunogenic component. In a further embodiment, the compositions of the invention comprise allogeneic, syngeneic, and/or autologous tumor cells and/or other glycoprotein antigens derived from tumor cells. In some embodiments, the compositions of the invention comprise a plurality of autologous tumor cells and/or other glycoprotein antigens derived from tumor cells, which may be the same or different. The autologous tumor cells and/or other glycoprotein antigens derived from tumor cells, may be administered separately or together. In one embodiment, the animal is human.

In one embodiment, the animal has a bacterial infection. In one embodiment, the compositions of the invention comprise bacterial cells and/or glycoprotein antigens derived from bacteria as the immunogenic component. In some embodiments, the compositions of the invention comprise a plurality of bacterial cells and/or glycoprotein antigens derived from bacteria. In some embodiments, the compositions of the invention comprise a plurality of bacterial cells and/or glycoprotein antigens derived from bacteria, which may be the same or different. In one embodiment, the animal is human.

In one embodiment, the animal has a viral infection. In one embodiment, the compositions of the invention comprise whole viruses, VLPs, and/or glycoprotein antigens derived from viruses as the immunogenic component. In some embodiments, the compositions of the invention comprise a plurality of whole viruses, VLPs, and/or glycoprotein antigens derived from viruses. In some embodiments, the compositions of the invention comprise a plurality of whole viruses, VLPs, and/or glycoprotein antigens derived from viruses, which may be the same or different. In one embodiment, the animal is human.

The compositions of the invention are generally administered in therapeutically effective amounts. For administration, the compositions of the invention can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose, and the like.

Suitable formulations for parenteral, subcutaneous, intradermal, intramuscular, oral, or intraperitoneal administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example ethyl oleate or triglycerides. Aqueous injection suspensions may contain substances which increase the viscosity of the suspension, include for example, sodium carboxymethyl cellulose, sorbitol, and/or dextran. Optionally, the suspensions may also contain stabilizers. Also, compositions can be mixed with immune adjuvants well known in the art such as Freund's complete adjuvant, inorganic salts such as zinc chloride, calcium phosphate, aluminum hydroxide, aluminum phosphate, saponins, polymers, lipids or lipid fractions (Lipid A, monophosphoryl lipid A), modified oligonucleotides, etc.

In addition to administration with conventional carriers, active ingredients may be administered by a variety of specialized delivery drug techniques which are known to those of skill in the art.

### EXAMPLES

The following examples are provided to further illustrate the advantages and features of the invention, but are not intended to limit the scope of this disclosure. All citations to patents and journal articles are hereby expressly incorporated by reference in their entireties.

### Example 1

### Synthesis of αGal (GlcNAc Containing Epitope) Amino Linker

### Synthesis of Compound 1

FIG. 5 shows the synthesis of αGal (GlcNAc containing epitope) amino linkers. As described in Agnihotri et al., 2005, acetic anhydride (85 ml, 900 mmol) and catalytic amount of DMAP (0.1 g) were added to a solution of D-galactose (27 g, 150 mmol) in pyridine (100 mL). After stirring over the weekend, the solvent was removed and the residue was portioned between EtOAc and H₂O. The organic phase was washed with brine and dried over anhydrous Na₂SO₄. After concentrated and dried under vacuum, the crude product was directly used for next step.

The crude intermediate was diluted by anhydrous CH₂Cl₂ (100 mL), followed by addition of p-toluenethiol (28 g; 225 mmol) in CH₂Cl₂ (50 mL). And additional BF₃-Et₂O (28 mL, 225 mmol) was added. After stirring overnight, the reaction was quenched by addition of aq NaHCO₃ and the mixture was extracted with EtOAc. The organic layer was washed with water, dried (Na₂SO₄), and concentrated under reduced pressure to give crude product.

A solution of crude peracetate thiolgalactoside (6.1 g, 13.4 mmol) and 0.5 M NaOMe (5.4 mL, 2.68 mmol) in MeOH (25 mL) was stirred at room temperature overnight. Then the reaction mixture was concentrated, and the residue was purified by flash column chromatography (5:1 CH₂Cl₂/MeOH) to give product (2.5 g, 65% from 3 steps).

### Synthesis of Compound 2

NaH (1.32 g, 52.4 mmol) was added to a solution of thiolglycoside 1 (2.5 g, 8.73 mmol) in anhydrous DMF (60 mL), followed by benzyl bromide (6.3 mL, 52.4 mmol) (Hsieh, et al., 2005). After stirring at room temperature overnight, the reaction was quenched by addition of MeOH (5 mL) and diluted by EtOAc. The reaction mixture was washed with H₂O, sat. NaHCO₃, brine, and dried over anhydrous Na₂SO₄. After concentration in vacuo, the residue was purified by flash column chromatography (10:1 Hex/EtOAc) to give product (4.4 g, 78%). CDCl₃ 400 MHz: 2.29 (s, 3H), 3.58-3.66 (m, 4H), 3.90 (t, 1H, J=9.3 Hz), 3.98 (d, 1H, J=2.6 Hz), 4.42 (d, 1H, J=11.6 Hz), 4.47 (d, 1H, J=11.6 Hz), 4.57-4.62 (m, 2H), 4.70-4.75 (m, 3H), 4.80 (d, 1H, J=10.0 Hz), 4.96 (d, 1H, J=11.6 Hz), 6.99 (d, 2H, J=8.0 Hz), 7.28-7.41 (m, 20H), 7.46 (d, 2H, J=8.0 Hz).

### Synthesis of Compound 3

The solution of thioglycoside 1 (24 g, 83.8 mmol) and Bu₂SnO (20.9 g, 83.8 mmol) in MeOH (200 mL) was refluxed under N₂ overnight (Xue et al., 2005). The reaction mixture was then concentrated. And the residue was azeotroped with toluene and dried under vacuum. To the crude intermediate was added DMF (200 mL), CsF (19.1 g, 125.7 mmol), NaI (18.8 g, 125.7 mmol) and 4-methoxbenzyl chloride (15.8 mL, 117.3 mmol) at −10° C. After being stirred at −10° C. for 1 hour, the reaction mixture was allowed to warm to room temperature and stirred for another 24 hours. Then the mixture was concentrated, and dried under vacuum. The residue was purified by flash column chromatography (1:2 hex/EtOAc) to give crude product.

To a solution of crude triol in pyridine (200 mL) at room temperature was added benzoyl chloride (43 mL, 0.37 mol) and catalytic amount of DMAP (200 mg). Then the reaction mixture was stirred at room temperature over the weekend. The solvent was removed and the residue was portioned between EtOAc and H₂O. The organic phase was washed with brine and dried over anhydrous Na₂SO₄. After concentration, the residue was purified by flash column chromatography (4:1 Hex/EtOAc) to give product (33 g, 55% from 3 steps). CDCl₃ 400 MHz: 2.31 (s, 3H), 3.69 (s, 3H), 3.80 (dd, 1H, J=9.4, 2.9 Hz), 4.13 (m, 1H), 4.40 (d, 1H, J=12.3 Hz), 4.46 (dd, 1H, J=11.5, 5.0 Hz), 4.57 (m, 1H), 4.60 (d, 1H, J=12.3 Hz), 4.78 (d, 1H, J=10.0 Hz), 5.47 (t, 1H, J=9.7 Hz), 5.89 (d, 1H, J=2.6 Hz), 6.57 (d, 2H, J=8.5 Hz), 7.00 (t, 4H, J=9.0 Hz), 7.42-7.49 (m, 8H), 7.58-7.62 (m, 3H), 7.98-8.12 (m, 6H).

### Synthesis of Compound 4

To a solution of thiolglycoside 3 (20 g, 27.8 mmol) in MeCN/H₂O (110 mL, 10:1) at room temperature was N-iodosaccharin (2.84 mg, 9.18 mmol) (Mandal et al., 2007). After stirring at room temperature for 5 hours, the solvent was diluted with CH₂Cl₂. The organic phase was washed with 20% Na₂S₂O₃, water and brine. After dried and concentrated, the residue was purified by flash column chromatography (3:1 Hex/EtOAc) to give product (10 g, 59%).

### Synthesis of Compound 5

To a solution hemi acetal 4 (9.7 g, 15.8 mmol) in anhydrous CH₂Cl₂ (60 mL) at room temperature was added trichloroacetonitrile (7.9 mL, 79.2 mmol) and DBU (1.18 mL, 7.9 mmol). The mixture was stirred for 2 hours at room temperature and concentrated. The residue was purified by flash column chromatography (4:1 Hex/EtOAc) to give product (10.3 g, 86%). CDCl₃ 400 MHz: 3.75 (s, 3H), 4.31 (dd, 1H, J=10.3, 3.1 Hz), 4.46 (dd, 1H, J=11.6, 5.1 Hz), 4.51-4.57 (m, 2H), 4.65 (t, 1H, J=6.2 Hz), 4.71 (d, 1H, J=12.1 Hz), 5.69 (dd, 1H, J=10.3, 3.3 Hz), 6.06 (d, 1H, J=2.1 Hz), 6.71 (d, 2H, J=8.5 Hz), 6.79 (d, 1H, J=3.3 Hz), 7.16 (d, 2H, J=8.5 Hz), 7.40-7.44 (m, 4H), 7.50 (t, 2H, J=7.7 Hz), 7.54-7.61 (m, 3H), 7.92 (d, 2H, J=7.5 Hz), 8.00 (d, 2H, J=7.5 Hz), 8.16 (d, 2H, J=7.5 Hz), 8.49 (s, 1H).

### Synthesis of Compound 6

To a solution of NaOMe (8.0 mL, 139 mmol; 25 wt % in methonal) in methanol (100 mL) was subsequentially added D-(+)-glucosamine hydrochloride (20 g, 93 mmol) and phthalic anhydride (13.9 g, 94 mmol) at room temperature (Nagorny et al., 2009). The resulting slurry was heated to reflux for 25 min whereupon a thick white precipitate was formed. The reaction was cooled to room temperature, filtered, and the residue was washed with cold methanol (2×50 mL). Upon drying, a white solid (25 g, 87%) was obtained that was used in the following transformation without further purification.

### Synthesis of Compound 7

To a suspension of GlcNPhth 6 (1.5 g, 4.85 mmol) in pyridine was added acetic anhydride (6.86 mL, 72.7 mmol) After stirring at room temperature overnight, the reaction mixture was diluted with EtOAc (20 mL), washed with saturated NH₄Cl, NaHCO₃, brine, and dried over Na₂SO₄, filtered, and concentrated. The residue was purified by flash column chromatography (3:2 Hex/EtOAc) to give product (1.8 g, 78%). CDCl₃ 400 MHz: 1.87 (s, 3H), 2.00 (s, 3H), 2.04 (s, 3H), 2.11 (s, 3H), 4.02 (m, 1H), 4.13-4.16 (m, 1H), 4.37 (dd, 1H, J=12.4, 4.2 Hz), 4.47 (dd, 1H, J=10.3, 9.2 Hz), 5.21 (t, 1H, J=9.7 Hz), 5.88 (dd, 1H, J=10.8, 9.7 Hz), 6.51 (d, 1H, J=9.0 Hz), 7.73-7.76 (m, 2H), 7.84-7.87 (m, 2H).

### Synthesis of Compound 8

Peracetate 7 (1.0 g, 2.1 mmol) was dissolved in 12 mL DCM and cooled to 0° C. then treated with 4 mL of a 33% solution of HBr in HOAc (Bennet et al., 2008). After 45 minutes the reaction was then brought to room temperature and stirred 45 minutes then treated with additional 4 mL of 33% HBr in HOAc. After 2 hours the reaction was diluted with 20 mL of CH₂Cl₂ and washed twice with aqueous NaHCO₃, twice with brine, dried (Na₂SO₄), filtered and concentrated in vacuo.

The crude glycosyl bromide, 2-azidoethanol (0.22 g, 2.51 mmol) and 4 Å MS (0.5 g) in anhydrous CH₂Cl₂ (10 mL) was stirred overnight. Then InCl₃ (185 mg, 0.84 mmol) was added, and the resultant mixture was stirred at room temperature overnight. Then the mixture was filtered through a celite pad, and concentrated. The residue was purified by flash column chromatography (3:2 Hex/EtOAc) to give product (0.6 g, 57%). CDCl₃ 400 MHz: 1.86 (s, 3H), 2.03 (s, 3H), 2.12 (s, 3H), 3.14-3.20 (m, 1H), 3.36-3.42 (m, 1H), 3.65 (ddd, 1H, J=11.5, 8.5, 3.2 Hz), 3.88 (ddd, 1H, J=10.2, 4.5, 2.4 Hz), 3.99-4.04 (m, 1H), 4.20 (dd, 1H, J=12.3, 2.2 Hz), 4.32 (dd, 1H, J=12.1, 4.8 Hz), 4.36 (dd, 1H, J=10.7, 8.5 Hz), 5.19 (t, 1H, J=9.6 Hz), 5.46 (d, 1H, J=8.5 Hz), 5.76 (dd, 1H, J=10.7, 9.2 Hz), 7.73 (dd, 2H, J=5.5, 3.0 Hz), 7.85 (dd, 2H, J=5.5, 3.0 Hz).

### Synthesis of Compound 9

Azido glycoside 8 (3.2 g, 6.3 mmol) was dissolved in 20 mL anhydrous MeOH, and followed by addition of 0.5M NaOMe in MeOH solution (2.5 mL, 1.3 mmol). After stirring for 3 hours, the reaction mixture was neutralized by acidic resin and concentrated. After being dried under a vacuum, the crude material was directly used for next step.

To a solution of crude triol (2.4 g, 6.3 mmol) and imidazole (0.6 g, 8.9 mmol) in anhydrous DMF (20 mL) at 0° C. was added TBDPSCl (1.8 mL, 7.0 mmol). The reaction mixture was then stirred at room temperature overnight, and then diluted by EtOAc. The organic phase was washed with sat. NH₄Cl, water, sat. NaHCO₃ and brine, and dried over anhydrous Na₂SO₄. After concentration, the residue was purified by flash column chromatography (3:2 Hex/EtOAc) to give product (3.2 g, 82% from 2 steps). CDCl₃ 400 MHz: 1.08 (s, 9H), 2.40 (d, 1H, J=4.5 Hz), 3.08-3.17 (m, 1H), 3.21 (d, 1H, J=2.2 Hz), 3.34 (ddd, 1H, J=11.8, 8.2, 3.6 Hz), 3.58-3.62 (m, 2H), 3.72 (t, 1H, J=9.0 Hz), 3.90-3.99 (m, 3H), 4.17 (dd, 1H, J=10.9, 8.4 Hz), 4.31-4.42 (m, 1H), 5.30 (d, 1H, J=8.4 Hz), 7.41-7.46 (m, 6H), 7.70-7.72 (m, 6H), 7.84-7.86 (m, 2H).

### Synthesis of Compound 10

Galactosyl trichloroacetimidate 5 (5.5 g, 7.27 mmol) and azido glycoside 9 (4.9 g, 7.99 mmol) were dried by coevaporation with anhydrous toluene and left under high vacuum. To the dried mixture was added 4 Å MS (2 g) and stirred in CH₂Cl₂ (30 mL) for 30 min at room temperature. The solution was cooled to −30° C. upon which TMSOTf (0.26 mL, 1.45 mmol) was added dropwise, and allowed to warm to room temperature over 3 hours. Upon completion, the reaction was quenched with sat. NaHCO₃ and filtered through a celite pad. The concentrated residue was purified by silica flash chromatography (3:1 Hex/EtOAc) to obtain disaccharide as a white powder (6.7 g, 76%). CDCl₃ 400 MHz: 0.86 (s, 9H), 3.10 (ddd, 1H, J=13.6, 5.2, 4.1 Hz), 3.27 (ddd, 1H, J=13.2, 7.9, 3.8 Hz), 3.45-3.49 (m, 2H), 3.70-3.82 (m, 6H), 3.98-4.08 (m, 2H), 4.19 (dd, 1H, J=10.4, 8.8 Hz), 4.28 (dd, 1H, J=11.4, 9.0 Hz), 4.42 (d, 1H, J=12.7 Hz), 4.56-4.64 (m, 2H), 4.77 (dd, 1H, J=11.7, 3.3 Hz), 4.88 (d, 1H, J=8.1 Hz), 5.21 (d, 1H, J=8.5 Hz), 5.58 (dd, 1H, J=9.7, 8.6 Hz), 5.89 (d, 1H, J=2.7 Hz), 6.62 (d, 2H, J=8.4 Hz), 7.04 (d, 2H, J=8.4 Hz), 7.19-7.29 (m, 5H), 7.34-7.61 (m, 15H), 7.66-7.85 (m, 7H), 8.08-8.14 (m, 4H).

### Synthesis of Compound 11

Disaccharide 10 (6.5 g, 5.37 mmol) was dissolved in pyridine (30 mL), followed by addition of Ac₂O (1.52 mL, 16.1 mmol) and catalytic amount of DMAP. After stirring at room temperature overnight, the mixture was diluted with EtOAc and washed with sat NH₄Cl, water, sat. NaHCO₃ and brine. The combined organic phase was dried and concentrated. The residue was purified by silica flash chromatography (2:1 Hex/EtOAc) to give product (5.2 g, 77%). CDCl₃ 400 MHz: 0.89 (s, 9H), 1.93 (s, 3H), 3.16 (ddd, 1H, J=13.4, 5.6, 3.7 Hz), 3.32 (ddd, 1H, J=13.2, 7.4, 3.5 Hz),3.42 (d, 1H, J=9.7 Hz), 3.54 (ddd, 1H, J=10.9, 7.6, 3.5 Hz), 3.70 (dd, 1H, J=10.1, 3.5 Hz), 3.74 (s, 3H), 3.78 (d, 1H, J=11.7 Hz), 3.86-3.92 (m, 2H), 3.97 (dd, 1H, J=8.3, 5.0 Hz), 4.24-4.44 (m, 4H), 4.62 (d, 1H, J=12.8 Hz), 4.68 (dd, 1H, J=11.5, 4.6 Hz), 5.02 (d, 1H, J=8.1 Hz), 5.36 (d, 1H, J=8.5 Hz), 5.51 (dd, 1H, J=9.9, 8.1 Hz), 5.82 (dd, 1H, J=10.7, 9.1 Hz), 5.86 (d, 1H, J=3.2 Hz), 6.60 (d, 2H, J=8.6 Hz), 7.04 (d, 2H, J=8.6 Hz), 7.19 (t, 3H, J=7.6 Hz), 7.24-7.32 (m, 3H), 7.36-7.87 (m, 19H), 8.12-8.17 (m, 4H).

### Synthesis of Compound 12

A solution of crude disaccharide 11 (4.0 g, 4.07 mmol) in 10% TFA/CH₂Cl₂ (20 mL) was stirred at room temperature for 3 hours. Then the mixture was diluted with EtOAc and quenched by NaHCO₃. The organic phase was washed with sat. NaHCO₃, brined, and dried. After concentration, the residue was purified by flash column chromatography (2:1 Hex/EtOAc) to give product (3.2 g, 88%). CDCl₃ 400 MHz: 0.99 (s, 9H), 1.90 (s, 3H), 2.66 (d, 1H, J=6.3 Hz), 3.18 (ddd, 1H, J=13.3, 5.5, 3.5 Hz), 3.34 (ddd, 1H, J=13.2, 7.7, 3.5 Hz), 3.49 (d, 1H, J=9.8 Hz), 3.56 (ddd, 1H, J=10.9, 7.7, 3.5 Hz), 3.90-3.96 (m, 2H), 4.01-4.09 (m, 3H), 4.25-4.32 (m, 2H), 4.39 (t, 1H, J=9.5 Hz), 4.64 (dd, 1H, J=11.5, 4.9 Hz), 5.12 (d, 1H, J=8.0 Hz), 5.31-5.38 (m, 2H), 5.71 (d, 1H, J=3.3 Hz), 5.83 (dd, 1H, J=10.8, 9.1 Hz), 7.28-7.30 (m, 2H), 7.35-7.43 (m, 4H), 7.45-7.52 (m, 5H), 7.58-7.63 (m, 4H), 7.70-7.85 (m, 10H), 8.10-8.15 (m, 4H).

### Synthesis of Compound 13

A suspension of donor 2 (3.2 g, 2.8 mmol), acceptor 12 (2.2 g, 3.4 mmol) and 4 Å MS (2 g) in anhydrous CH₂Cl₂ (30 mL) was stirred at room temperature for 30 min. Then the resulting mixture was cooled to −20° C., followed by addition of NIS (0.95 g, 4.2 mmol) and TfOH (25 μl, 0.28 mmol). The reaction mixture was stirred at −20° C. for 3 hours, and then the reaction was quenched by addition of sat. Na₂S₂O₃ and filtered through a celite pad. After concentration, the residue was purified by flash column chromatography (3:1 hex/EtOAc) to give product (3.43 g, 73%).

### Synthesis of Compound 14

A solution of benzyl glycoside 13 (3.4 g, 2.05 mmol) in anhydrous THF (20 mL) was added 1 M TBAF solution (6.2 mL, 6.2 mmol). After stirring at room temperature overnight, the mixture was concentrated and dried under vacuum. The residue was then dissolved in ethanol/toluene (30 mL, 3:2), followed by addition of NH₂NH₂—H₂O (3.0 mL, 61.6 mmol). After refluxed overnight, the solvent was removed and dried under vacuum. The crude product was used for next step directly.

### Synthesis of Compound 15

A solution of crude amine 14 in pyridine (20 mL) was added Ac₂O (4.05 mL 42.9 mmol) and catalytic amount of DMAP. The resulting mixture was stirred at room temperature overnight, and was then diluted with EtOAc. The organic phase was washed with sat. NH₄Cl, water, sat. NaHCO₃ and brine, and dried over Na₂SO₄. After concentration, the residue was purified by flash column chromatography (1:4 hex/EtOAc) to give product (1.6 g, 63% from 3 steps). CDCl₃ 400 MHz: 1.81 (s, 3H), 1.93 (s, 3H), 1.97 (s, 3H), 2.04 (s, 3H), 2.06 (s, 3H), 2.07 (s, 3H), 3.27 (ddd, 1H, J=13.3, 4.8, 3.3 Hz), 3.44-3.52 (m, 3H), 3.62-3.69 (m, 3H), 3.73-3.87 (m, 5H), 3.96-4.15 (m, 6H), 4.35 (d, 1H, J=7.9 Hz), 4.40 (d, 1H, J=11.8 Hz), 4.47-4.55 (m, 4H), 4.63 (d, 1H, J=11.5 Hz), 4.70 (dd, 2H, J=11.5, 5.5 Hz), 4.82 (d, 1H, J=11.8 Hz), 4.91 (d, 1H, J=11.5 Hz), 5.05-5.12 (m, 3H), 5.44 (d, 1H, J=2.9 Hz), 5.71 (d, 1H, J=9.4 Hz), 7.24-7.37 (m, 20H).

### Synthesis of Compound 16

A mixture of azide glycoside 15 (1.5 g, 1.27 mmol) and 0.5 M NaOMe (1.0 mL, 0.51 mmol) in MeOH (20 mL) was stirred at 50° C. for 4 hours (Arranz-Plaza et al., 2002). Then the reaction mixture was neutralized by acidic resin, and concentrated to give product (1.1 g, 89%).

The crude intermediate (0.5 g, 0.51 mmol) was dissolved in EtOH/HCl (30/0.2 mL), followed by addition of Pd/C (400 mg). The reaction mixture was shaken under 50 psi H2 overnight. Then the mixture was filtered through celite, and neutralized by NaOH solution. After concentration, the residue was purified by bio-gel P2 column to give product (0.3 g, 45%).

D₂O 400 MHz: 2.06 (s, 3H), 3.17-3.29 (m, 2H), 3.65-4.07 (m, 18H), 4.19-4.22 (m, 2H), 4.55 (d, 1H, J=7.8 Hz), 4.60 (d, 1H, J=8.0 Hz), 5.15 (d, 1H, J=3.8 Hz).

### Example 2

### Synthesis of αGal (Glc Containing Epitope) Amino Linker

### Synthesis of Compound 17

FIG. 6 shows the synthesis of a αGal (Glc containing epitope) amino linker. The mixture of lactose (30 g, 87.6 mmol), acetic acid (102 mL, 1.05 mol) and DMAP (100 mg) in pyridine (150 mL) was stirred at room temperature over the weekend. The residue was diluted in EtOAc, washed with 1 N HCl, H₂O, saturated NaHCO₃ (aq), brine and dried over anhydrous Na₂SO₄. After concentration and drying under a vacuum, the crude product was directly used for next step.

### Synthesis of Compound 18

To a cooled (ice-water), stirred solution of peracetylated lactose 17 (20.0 g, 29.5 mmol), 2-N-phthalimide ethanol (6.76 g, 35.4 mmol, 1.2 eq) in dichloromethane (150 mL) was added BF₃-etherate (18.5 mL, 147 mmol). The reaction mixture was stirred for 1 hour at 0° C., then 12 hrs at room temperature under an N₂ atmosphere. Additional BF3-etherate (10 mL) was added, and the mixture was stirred overnight. Then the reaction was quenched by addition of sat. NaHCO₃, and washed with saturated NaHCO₃ and brine. After being dried over anhydrous Na₂SO₄, the filtrate was evaporated under reduced pressure and the residue was purified by column chromatography (3:2 EtOAc/Hex) to give product (17 g, 71%). CDCl₃ 400 MHz: 1.85 (s, 3H), 1.95 (s, 3H), 1.99 (s, 3H), 2.03 (s, 3H), 2.05 (s, 3H), 2.11 (s, 3H), 2.13 (s, 3H), 3.54-3.58 (m, 1H), 3.71-3.91 (m, 6H), 3.97-4.03 (m, 2H), 4.06-4.12 (m, 2H), 4.39-4.47 (m, 3H), 4.83 (t, 1H, J=8.1 Hz), 4.93 (dd, 1H, J=10.4, 2.9 Hz), 5.06-5.14 (m, 2H), 5.32 (d, 1H, J=2.3 Hz), 7.71-7.73 (m, 2H), 7.83-7.85 (m, 2H).

### Synthesis of Compound 19

Phthalimide glycoside 18 (17 g, 1.9 mmol) was dissolved in 100 mL anhydrous MeOH, and followed by addition of 25% NaOMe in MeOH (0.24 mL, 4.2 mmol). The reaction mixture was stirred for 3 hours until a lot of white precipitate formed. The precipitate was collected by filtration, and washed with MeOH twice (30 mL×2). After being dried under vacuum, the product (7 g, 65%) was directly used for next step. D₂O 400 MHz: 3.21 (t, 1H, J=8.5 Hz), 3.49-3.78 (m, 10H), 3.81-3.96 (m, 4H), 4.05-4.09 (m, 1H), 4.36 (d, 1H, J=7.8 Hz), 4.40 (d, 1H, J=7.9 Hz), 7.78-7.82 (m, 4H).

### Synthesis of Compound 20

The solution of phthalimide glycoside 19 (6.5 g, 12.6 mmol) and Bu₂SnO (4.7 g, 18.9 mmol) in MeOH (100 mL) was refluxed under N₂ overnight (Xue et al., 2005). The reaction mixture was then concentrated. Then the residue was azeotroped with toluene and dried under vacuum. To the crude intermediate was added DMF (60 mL), CsF (2.9 g, 18.9 mmol), NaI (2.8 g, 18.9 mmol) and 4-methoxbenzyl chloride (2.4 mL, 17.7 mmol) at −10° C. After being stirred at −10° C. for 1 hour, the reaction mixture was allowed to warm to room temperature and stirred for another 24 hours. The mixture was then concentrated, and dried under vacuum. The crude product was used for next step directly.

### Synthesis of Compound 21

To a solution of PMB protected glycoside 20 in pyridine (6 mL) at room temperature was added Ac₂O (0.86 mL, 8.8 mmol). Then the reaction mixture was stirred at room temperature overnight. The solvent was removed and the residue was portioned between EtOAc and H₂O. The organic phase was washed with brine and dried over anhydrous Na₂SO₄. After being concentrated, the residue was purified by flash column chromatography (1:1 Hex/EtOAc) to give product (0.35 g, 63%). CDCl₃ 400 MHz: 1.84 (s, 3H), 1.99 (s, 6H), 2.08 (s, 6H), 2.13 (s, 3H), 3.43 (dd, 1H, J=10.0, 3.4 Hz), 3.56 (dq, 1H, J=7.9, 3.3, 2.7 Hz), 3.67 (dd, 1H, J=9.9, 8.9 Hz), 3.70-3.76 (m, 1H), 3.80 (s, 4H), 3.85-3.91 (m, 2H), 3.94-4.02 (m, 2H), 4.08 (dd, 2H, J=6.7, 2.1 Hz), 4.28 (d, 1H, J=11.8), 4.31 (d, 1H, J=8.0 Hz), 4.36 (dd, 1H, J=11.8, 2.1 Hz), 4.45 (d, 1H, J=7.8 Hz), 4.58 (d, 1H, J=11.8 Hz), 4.82 (dd, 1H, J=9.5, 7.8 Hz), 4.96 (dd, 1H, J=10.0, 8.0 Hz), 5.10 (t, 1H, J=9.2 Hz), 5.42 (dd, 1H, J=3.5, 1.2 Hz), 6.85 (d, 2H, J=8.7 Hz), 7.14 (d, 2H, J=8.7 Hz), 7.71 (dd, 2H, J=5.5, 3.0 Hz), 7.83 (dd, 2H, J=5.5, 3.1 Hz).

### Synthesis of Compound 22

A solution of crude disaccharide 21 (0.35 g, 0.39 mmol) in 10% TFA/CH₂Cl₂ (6 mL) was stirred at room temperature for 3 hours. Then the mixture was diluted with EtOAc and quenched by NaHCO₃. The organic phase was washed with saturated NaHCO₃, brined and dried. After being concentrated, the residue was purified by flash column chromatography (1:3 Hex/EtOAc) to give product (0.3 g, 99%). CDCl₃ 400 MHz: 1.84 (s, 3H), 1.99 (s, 3H), 2.07 (s, 3H), 2.10 (s, 3H), 2.11 (s, 3H), 2.15 (s, 3H), 2.58 (brs, 1H), 3.55-3.62 (m, 1H), 3.66-3.84 (m, 4H), 3.89 (dt, 2H, J=7.9, 6.1 Hz), 3.96-4.17 (m, 4H), 4.37 (d, 1H, J=7.9 Hz), 4.39-4.52 (m, 2H), 4.82-4.85 (m, 2H), 5.11 (t, 1H, J=9.3 Hz), 5.27 (dd, 1H, J=3.6, 1.2 Hz), 7.72 (dd, 2H, J=5.5, 3.0 Hz), 7.84 (dd, 2H, J=5.5, 3.0 Hz).

### Synthesis of Compound 23

A suspension of donor 2 (2.22 g, 3.44 mmol), acceptor 22 (2.2 g, 2.87 mmol) and 4 Å MS (5200 mg) in anhydrous CH₂Cl₂ (25 mL) was stirred at room temperature for 30 min. Then the resulting mixture was cooled to −20° C., followed by addition of NIS (1.29 g, 5.7 mmol) and TfOH (51 μl, 0.57 mmol). The reaction mixture was stirred at −20° C. for 2 hours, and then the reaction was quenched by addition of saturated Na₂S₂O₃ and filtered through a celite pad. After being concentrated, the residue was purified by flash column chromatography (1:1 hex/EtOAc) to give product (3.1 g, 84%). CDCl₃ 400 MHz: 1.80 (s, 3H), 1.84 (s, 3H), 1.91 (s, 3H), 1.96 (s, 3H), 2.06 (s, 3H), 2.07 (s, 3H), 3.49 (d, 2H, J=6.5 Hz), 3.54-3.58 (m, 1H), 3.63 (t, 1H, J=6.5 Hz), 3.67 (t, 1H, J=9.4 Hz), 3.73-3.84 (m, 5H), 3.85-3.92 (m, 2H), 3.94-4.03 (m, 5H), 4.28 (d, 1H, J=7.9 Hz), 4.37 (dd, 1H, J=11.9, 2.1 Hz), 4.39 (d, 1H, J=11.8 Hz), 4.43-4.52 (m, 3H), 4.62 (d, 1H, J=11.6 Hz), 4.65-4.72 (m, 2H), 4.77-4.85 (m, 2H), 4.90 (d, 1H, J=11.3 Hz), 5.00-5.16 (m, 3H), 5.41 (d, 1H, J=2.6 Hz), 7.18-7.40 (m, 20H), 7.71 (dd, 2H, J=5.5, 3.1 Hz), 7.84 (dd, 2H, J=5.5, 3.1 Hz).

### Synthesis of Compound 24

A suspension of trisaccharide 23 (3.1 g, 2.4 mmol) and Pd(OH)₂/C (20%, 0.6 g) in MeOH/HCl (30/0.3 mL) was shaken under 50 psi H2 overnight. After being filtered through a celite pad, the solvent was removed under reduced pressure. The residue was redissolved in EtOH/toluene (45 mL, 3:2), followed by addition of NH₂NH₂—H₂O (3.5 mL, 72 mmol). The mixture was refluxed overnight. Then the mixture was concentrated, and the residue was purified by bio-gel P2 column to give product (900 mg, 68%). D₂O 400 MHz: 2.84-3.07 (m, 2H), 3.34 (td, 2H, J=7.7, 2.5 Hz), 3.55-3.87 (m, 12H), 3.90-4.05 (m, 4H), 4.16-4.19 (m, 2H), 4.50 (d, 2H, J=7.9 Hz), 5.13 (d, 1H, J=3.8 Hz).

### Example 3

### Synthesis of Gal(α1-3)Gal(β1-4)Glc-Aminooxy Linkers

FIG. 7 shows the synthesis of Gal(α1-3)Gal(β1-4)Glc-aminooxy linkers.

### Synthesis of Compound 25

To a stirred solution of N-Boc-aminooxyacetic acid (0.500 g, 2.6 mmol) in ethyl acetate/dioxane (1:1, 10 mL) cooled on an ice bath were added N-hydroxysuccinimide (0.310 g, 2.7 mmol) and DCC (0.563 g, 2.7 mmol) (Foillard et al., 2008). The resulting mixture was stirred at room temperature for 5 hours and was then filtered through a pad of Celite, and the filtrate was concentrated under vacuum. The obtained residue was redissolved in ethyl acetate (35 mL) and washed with 5% aqueous NaHCO₃ (3×5 mL), water (2×10 mL), and brine (10 mL). The organic phase was dried over Na₂SO₄ and evaporated in vacuo to give product as a white solid (0.68 g, 90%).

### Synthesis of Compound 26

To a solution of amino linker 24 (30 mg, 55 umol) in DMSO (1.0 mL) was added activated acid 25 (19 mg, 66 umol) and Et₃N (11.5 μl, 82 umol). After been stirred at room temperature for 2 hours, the product was precipitated with acetone/ether (1:2, 10 mL). And the residue was washed with acetone/ether (1:1, 10 mL), and dried in vacuo. The crude product was purified by flash column chromatography (32:68 MeOH/EtOAc) to give product (55 mg, 84%). D₂O 400 MHz: 1.46 (s, 9H), 3.31-3.36 (m, 2H), 3.44-3.88 (m, 14H), 3.90-4.04 (m, 4H), 4.16-4.19 (m, 2H), 4.37 (s, 2H), 4.46-4.55 (m, 2H), 5.13 (d, 1H, J=3.8 Hz).

### Synthesis of Compound 27 (CAL-a08)

Boc protected linker 26 (30 mg, 42 umol) in TFA/CH₂Cl₂ (1 mL, 4:6) was stirred at room temperature for 1 hour. Then the solvent was removed under reduced pressure, and the residue was dried under vacuum to give final product (25 mg, 97%). D₂O 400 MHz: 3.26-3.36 (m, 2H), 3.44-3.88 (m, 14H), 3.90-4.04 (m, 4H), 4.16-4.19 (m, 2H), 4.44-4.53 (m, 2H), 4.61 (s, 2H), 5.13 (d, 1H, J=3.8 Hz).

### Synthesis of Compound 28

5-(Boc-amino)pentanoic acid (0.5 g, 2.30 mmol) was dissolved in 20 mL of dichloromethane, followed by addition of N-Hydroxysuccinimide (291 mg, 2.53 mmol), and N,N′-dicyclohexylcarbodiimide (570 mg, 2.76 mmol), and catalytic amount of 4-dimethylamiopryidine were added (Mao et al., 2012). After being stirred for 2 hours at room temperature, the solution was filtered to remove precipitation, dried and evaporated under reduced pressure to yield light yellow oil. The white powder was used for the next step without further purification.

### Synthesis of Compound 29

To a solution of amino linker 24 (50 mg, 91 mmol) in DMSO (2.0 mL) was added activated acid 28 (47 mg, 137 umol) and Et₃N (25 μl, 183 umol). After being stirred at room temperature overnight, the product was precipitated with acetone/ether (1:2, 10 mL). Then the residue was washed with acetone/ether (1:1, 10 mL), and dried in vacuo to give product (58 mg, 85%). D₂O 400 MHz: 1.42 (s, 9H), 1.45-1.52 (m, 2H), 1.54-1.66 (m, 2H), 2.27 (t, 2H, J=7.3 Hz), 3.06 (t, 2H, J=3.7 Hz), 3.25-3.52 (m, 3H), 3.51-3.89 (m, 13H), 3.89-4.03 (m, 4H), 4.13-4.23 (m, 2H), 4.48-4.52 (m, 2H), 5.14 (d, 1H, J=3.8 Hz).

### Synthesis of Compound 30

Boc protected linker 29 (44 mg, 58 umol) in TFA/CH₂Cl₂ (2 mL, 4:6) was stirred at room temperature for 1 hour. Then the solvent was removed under reduced pressure, and the residue was purified by bio-gel P2 column (2% NH₄OH/H₂O) to give final product (46 mg, 92%). D₂O 400 MHz: 1.63-1.67 (m, 4H), 2.22-2.36 (m, 2H), 2.95-2.99 (m, 2H), 3.29-3.35 (m, 1H), 3.41-3.45 (m, 2H), 3.54-3.88 (m, 13H), 3.89-4.04 (m, 4H), 4.16-4.18 (m, 2H), 4.47-4.51 (m, 2H), 5.143 (d, 1H, J=3.9 Hz).

### Synthesis of Compound 31

To a solution of amino linker 30 (35 mg, 54 umol) in DMSO (1.0 mL) was added activated acid 25 (23 mg, 81 umol) and Et₃N (15 μl, 108 umol). After being stirred at room temperature for 2 hours, the product was precipitated with acetone/ether (1:2, 10 mL). And the residue was washed with acetone/ether (1:1, 10 mL), and dried in vacuo. The crude product was purified by bio-gel P2 column to give product (25 mg, 56%). D₂O 400 MHz: 1.41-1.66 (m, 6H), 1.47 (s, 9H), 2.29 (t, 2H, J=7.1 Hz), 3.23-3.50 (m, 5H), 3.56-3.89 (m, 11H), 3.91-4.04 (m, 4H), 4.15-4.24 (m, 2H), 4.35 (s, 2H), 4.49 (d, 1H, J=7.9 Hz), 4.51 (d, 1H, J=7.9 Hz), 5.14 (d, 1H, J=3.9 Hz).

### Synthesis of Compound 32 (CAL-a11)

Boc protected linker 31 (22 mg, 27 umol) in TFA/CH₂Cl₂ (1 mL, 4:6) was stirred at room temperature for 1 hour. Then the solvent was removed under reduced pressure, and the residue was dried under vacuum to give final product (14 mg, 81%). D₂O 400 MHz: 1.43-1.68 (m, 4H), 2.27 (t, 2H, J=7.0 Hz), 3.19-3.34 (m, 3H), 3.34-3.49 (m, 2H), 3.53-4.87 (m, 13H), 3.89-4.06 (m, 4H), 4.15-4.19 (m, 2H), 4.46-4.50 (m, 2H), 4.58 (s, 2H), 5.12 (d, 1H, J=3.8 Hz).

### Example 4

### Synthesis of Gal(α1-3)Gal(β1-4)GlcNAc-Aminooxy Linkers

FIG. 8 shows the synthesis of Gal(α1-3)Gal(β1-4)GlcNAc-aminooxy linkers.

### Synthesis of Compound 33

To a solution of amino linker 16 (48 mg, 82 mmol) in DMSO (1.5 mL) was added activated acid 28 (38 mg, 122 umol) and Et₃N (23 uL, 163 μmol). After been stirred at room temperature overnight, the product was precipitated with acetone/ether (1:2, 10 mL). And the residue was washed with acetone/ether (1:1, 10 mL), and dried in vacuo to give product (33 mg, 51%) D₂O 400 MHz: 1.42 (s, 9H), 1.44-1.50 (m, 2H), 1.50-1.62 (m, 2H), 2.03 (s, 3H), 2.26 (t, 2H, J=7.4 Hz), 3.07 (t, 2H, J=6.7 Hz), 3.30-3.43 (m, 2H), 3.50-4.08 (m, 18H), 4.17-4.20 (m, 2H), 4.52-4.55 (m, 2H), 5.14 (d, 1H, J=3.8 Hz).

### Synthesis of Compound 34

Boc protected linker 33 (33 mg, 42 umol) in TFA/CH₂Cl₂ (2 mL, 4:6) was stirred at rt for 1 h. Then the solvent was removed under reduced pressure, and the residue was purified by bio-gel P2 column (2% NH₄OH/H₂O) to give final product (28 mg, 97%). D₂O 400 MHz: 1.63-1.65 (m, 4H), 2.01 (s, 3H), 2.26-2.30 (m, 2H), 2.96-2.99 (m, 2H), 3.34-3.37 (m, 2H), 3.58-4.00 (m, 17H), 4.15-4.19 (m, 2H), 4.50-4.53 (m, 2H), 5.12 (d, 1H, J=3.6 Hz).

### Synthesis of Compound 35

The solution of acid (12 mg, 61 umol), TSTU (25 mg, 81 umol) and Et₃N (14 uL, 102 umol) in DMF (1 mL) was stirred at rt for 2 h. Then the mixture was added to a solution of amino linker 34 (28 mg, 41 umol) in DMSO (1 mL). After been stirred at room temperature for 2 h, the mixture was concentrated under vacuo to final volume 1.5 mL, and then was precipitated with acetone/ether (1:2, 10 mL). And the ppt was washed with acetone/ether (1:1, 10 mL), and dried in vacuo. The ppt was washed with CH₂Cl₂, and centrifuged to give final product after dried in vacuo (27 mg, 77%). D₂O 400 MHz: 1.38-1.68 (m, 6H), 1.46 (s, 9H), 2.02 (s, 3H), 2.26 (t, 2H, J=6.8 Hz), 3.27 (t, 2H, J=6.5 Hz), 3.34-3.37 (m, 2H), 3.53-4.06 (m, 16H), 4.16-4.20 (m, 2H), 4.34 (s, 2H), 4.51-4.54 (m, 2H), 5.13 (d, 1H, J=3.8 Hz).

### Synthesis of Compound 36 (CAL-aN11)

Boc protected linker 35 (25 mg, 29 umol) in TFA/CH₂Cl₂ (1 mL, 4:6) was stirred at rt for 1 h. Then the solvent was removed under reduced pressure, and the residue was dried under vacuum to give final product (20 mg, 90%). D₂O 400 MHz: 1.52-1.66 (m, 4H), 2.03 (s, 3H), 2.24-2.29 (m, 2H), 3.25-3.29 (m, 2H), 3.34-3.38 (m, 2H), 3.59-4.02 (m, 16H), 4.17-4.19 (m, 4H), 4.52-4.55 (m, 2H), 4.58 (s, 2H), 5.14 (d, 1H, J=3.9 Hz).

### Example 5

### Synthesis of Rhamnose Aminooxy Linkers

FIG. 9 shows the synthesis of rhamnose aminooxy linkers. Rhamnose aminooxy linkers are synthesized as described in Example 1. Treatment of L-rhamnose with acetic anhydride in pyridine gives peracetylated intermediate quantitatively. The following glycosylation with N-(2-Hydroxyethyl)phthalimide promoted by BF₃-Et₂O leads to fully protected rhamnose phthalimide linker. Deprotection of both acetyl and phthalimide groups is achieved by the treatment with hydrazine hydrate in methanol. The reaction between rhamnose amino linker and NHS-activated aminooxy precursor (compound 25) in the presence of Et₃N results in N-Boc protected rhamnose aminooxy linker. The final treatment with 40% TFA in CH₂Cl₂ provides rhamnose aminooxy linker #1.

A spacer elongation reaction between rhamonse amino linker and NHS-activated 5-(Boc-amino)valeric acid (compound 28) yields a N-Boc protected rhamnose amino linker. Deprotection of the Boc group is accomplished by using 40% TFA in CH₂Cl₂. Amidation between the amino linker and compound 25 provides N-Boc protected aminooxy linker, which undergoes deprotection with 40% TFA in CH₂Cl₂ to yield rhamnose aminooxy linker #2.

### Example 6

### Synthesis of Forssman Disaccharide Aminooxy Linkers

FIG. 10 shows the synthesis of Forssman disaccharide aminooxy linkers. Synthesis of Forssman disaccharide aminooxy linkers is described in Example 2. After activation by N-iodosuccinimide (NIS) and trifluoromethanesulfonic acid (TfOH), Forssman disaccharide p-toluenethiol donor (Chen, 2010) reacts with N-(2-Hydroxyethyl)phthalimide to give N-phthalimide protected linker. Deprotection of benzylidene group using p-toluenesulfonic acid (p-TsOH), followed by zinc reduction in a mixture of THF/Ac₂O/AcOH yields the N-phthalimide diol linker. Deprotection of the remaining acetyl protected hydroxyl groups is accomplished by the treating starting material with hydrazine hydrate in methanol. The reaction between the Forssman disaccharide amino linker and the NHS-activated aminooxy precursor (compound 25) in the presence of Et₃N results in N-Boc protected aminooxy linker A final deprotection with 40% TFA in CH₂Cl₂ provides Forssman disaccharide aminooxy linker #1.

Using the same strategy as for rhamnose aminooxy linker synthesis described above in Example 5, the spacer elongation reaction between the Forssham disaccharide amino linker and the NHS-activated 5-(Boc-amino)valeric acid (compound 28) yields the N-Boc protected amino linker. Deprotection of the N-Boc group is accomplished with 40% TFA in CH₂Cl₂. Amidation between amino linker and compound 25 provides N-Boc protected aminooxy linker, which is then treated with 40% TFA in CH₂Cl₂ to give Forssman disaccharide aminooxy linker #2.

### Example 7

### Carbohydrate-Specific Modification of Recombinant HA (rHA) Using a Combination of NaIO₄ and αGal Aminooxy Linker 27

### Oxidation of rHA by NaIO₄

100 μg of lyophilized rHA (PR8 H1N1) powder was washed with 0.1 M NaOAc by ultrafiltration at 14,000×g for 15 min using 10 kDa cut-off centrifugal filter device (EMD Millipore, Billerica, Mass.) for three times. After washing, 0.1 M NaOAc buffer (pH 5.5) was added to make final volume at 100 μl. To this protein solution was then added 22 μl of freshly prepared NaIO4 solution (10 mg/mL) to get a final NaIO₄ concentration at 10 mM. After shaking for 30 min at room temperature with protection from light, the mixture was washed with 1×PBS (GIBCO DPBS) by ultrafiltration at 14,000×g for 15 min using 10 kDa cut-off centrifugal filter device for three times to remove all reagents. The oxidized protein was prepared as a final volume at 100 μl in 0.1 M NaOAc buffer (pH 5.5) for the next step.

### Conjugation

To the oxidized rHA solution from previous step was added 10 μl of αGal aminooxy linker (20 mg/mL) and 0.5 μl of aniline. The reaction mixture was shaken overnight at 4° C., and then was washed with 1×PBS by ultrafiltration at 14,000×g for 15 min using 10 kDa cut-off centrifugal filter device for three times to remove all reagents. The final conjugate was stored as a 100 μl solution in 1×PBS.

### Characterization of αGal-rHA Conjugate

FIG. 11 shows (A) the SDS-PAGE silver staining analysis and (B) anti-αGal western blot of different rHA before and after modification. Lane 1 contains the original, unmodified rHA, and lane 2 contains oxidized rHA with αGal aminooxy linker conjugation. Lane 2 shows a distinct migration, indicating that the αGal epitope was successfully conjugated to the oxidized protein. This was confirmed by the binding of the chicken polyclonal anti-αGal antibody to the contents of lane 2. The Western Blot was performed using chicken polyclonal anti-αGal as the primary antibody at 1:5000 dilution with a secondary antibody of AP-Rabbit anti-Chicken/Turkey IgG (Life Technologies Corp.) at 1:2000 dilution.

### Deglycosylation Assay

Original, unmodified rHA, aminooxy linker modified rHA, and NHS-activated linker modified rHA were included in this assay in order to confirm the selectivity of modification site and the activity on the different substrates of the glycosidases PNGase-F and Endo-H.

Deglycosylation by PNGase F treatment consisted of combining 16 μg of each glycoprotein sample, 4.4 μl of 10× Glycoprotein Denaturing Buffer and H₂O (if necessary) to make a 44.4 μl total reaction volume. The glycoprotein was denatured by heating at 95° C. for 10 minutes. The total reaction volume was adjusted to 30 μl by adding, 20 μl of denatured sample, 3 μl of 10×G7 Reaction Buffer, 3 μl of 10% NP-40, 2 μl of H₂0 and 2 μl PNGase to the mixture. The reaction was then incubated at 37° C. for 1 hour.

Deglycosylation by Endo-H treatment consisted of combining 16 μg of each glycoprotein sample, 4.4 μl of 10× Glycoprotein Denaturing Buffer, and H₂O (if necessary) to make a 44.4 μl total reaction volume. The glycoprotein was denatured by heating at 95° C. for 10 minutes. The total reaction volume was adjusted to 30 μl by adding 20 μl of denatured sample, 3 μl of 10×G5 Reaction Buffer, 5 μl of H₂O and 2 μl Endo-H. The reaction was then incubated at 37° C. for 1 hour.

FIG. 12 shows the SDS-PAGE (A) and anti-αGal western blot (B) assay for rHA (lanes 1 and 4), rHA modified on the lysine residues with an αGal linker (lanes 2 and 5) and rHA modified on the carbohydrate residues with an αGal linker of the present invention (lanes 3 and 6), after treatment with the glycosidase PNGaseF (lanes 1 to 3) or EndoH (lanes 4 to 6). Different migration patterns in these two lanes after treatment with different enzymes demonstrated that the different enzymes exhibited different degrees of deglycosylation based on their substrate selectivity and activity. PNGase F caused more deglycosylation than Endo-H in all three samples. The figure shows that modification of the HA glycoprotein on lysine residues with αGal-linkers activated with NHS results in epitopes that cannot be removed by treatment with PNGaseH or EndoH. Conversely, modification of the HA glycoprotein by addition of αGal linkers on pre-existing carbohydrate moieties via aminoxy activation results on αGal epitopes that can be removed by treatment with PNGaseF and EndoH. These figures also show that the aminooxy linker modified samples lost more αGalsignal under a higher degree of deglycosylation. This result confirmed that the type of αGal modification of the present invention targets glycosylation sites, but not any other site.

### Example 8

### Terminal Galactose-Specific Modification of H1N1 VLP Using a Combination of Galactose Oxidase and αGal Aminooxy Linker 32 (CAL-a11)

### Oxidation of H1N1 VLP by Galactose Oxidase

Ten microliters of catalase (10 U/μl) and 5 μl of GO (500 U/ml; SigmaG7907-150UN) were added to 170 μl of influenza VLP (PR8 H1N1) in 1×PBS. After Incubation at 37° C. for 2 hours, the mixture was ultra-centrifuged at 21000 g for 30 minutes to pellet VLP. The supernatant was discarded, and the pellet was resuspended in 200 μl 1×PBS, and ultra-centrifuged again. The supernatant was discarded and the pellets were resuspended with 150 μl 0.1 M NaOAc buffer.

### Conjugation

Ten microliters of αGal aminooxy linker CAL-a11 (20 mg/mL) and 0.75 μl of aniline was added to the oxidized VLP suspension from the previous step. The reaction mixture was shaken overnight at 4° C., and then ultra-centrifuged at 21000 g for 30 minutes to pellet the VLPs. The supernatant was discarded, and the pellet was resuspended in 200 μl 1×PBS, and ultra-centrifuged again. The ultra-centrifugation was repeated two more times. The final pellet was resuspended in 80 μl of 1×PBS (containing 4% sucrose) and stored at −20° C.

### Characterization of αGal-VLP Conjugate

### SDS-PAGE and Western Blot

FIG. 13 shows the (A) SDS-PAGE, (B) anti-HA western blot, and (C) anti-αGal western blot assays for this modification. Approximately 400 ng of HA protein was loaded in each lane. Lane 1 contains the original, unmodified VLP sample, lane 2 contains the VLP oxidized by GO only, and lane 3 contains the product after conjugation of the VLPs with αGal aminooxy linker. Both SDS-PAGE and anti-HA western blot indicate the successful addition of αGal onto VLP, since lane 3 shows significant shift comparing to lanes 1 and 2. The binding demonstrated in the anti-αGal western blot (C) further confirms that αGal is successfully added to the VLPs.

### Hemagglutination Assay.

An essential feature of influenza hemagglutinin protein is the ability of the protein to bind to red blood cells as a trimeric or oligomeric molecule. The functional features of the hemagglutinin protein that allow it to form oligomers and trimers are essential for its ability to induce a strong vaccine response (Wei et al., 2008; Welsh et al., 2012; Du et al., 2013). In this experiment, a 1:100 dilution of each sample was prepared as stock solution before the assay. In a 96-well plate, stock solutions were added to the first well and serial 2-fold dilutions in 1×PBS were performed along each row to get 100 μl final volume in each well. The last column was PBS only as a negative control. After the samples had been diluted, 50 μl of the washed turkey red blood cells (RBCs) (0.5% in 1×PBS) was added to each well. The plate was tapped on the bottom to mix, and then incubated at room temperature for 1 hour. Hemagglutination occurs when the VLPs binds to the RBCs, causing the cells to fall uniformly over the bottom of a round bottom plate. If there is no hemagglutination, the RBCs will settle into the bottom of the well, creating a red button of cells.

As shown in FIG. 14, the original, unmodified VLPs (group #1, rows 1 & 2) induced hemagglutination down to a 1:64 dilution. Oxidized VLPs (with GO) (group #2, rows 3 & 4) and aminooxy linker modified VLPs (group #3, rows 5 and 6) have similar HA activity at a dilution of 1:32, indicating a minimal loss of structure. However, the HA activity of modified VLPs that were linked using typical N-hydroxysuccinimide chemistry (group #4, rows 7 & 8) lost a significant amount of activity (having HA activity to only 1:2). This result indicates that the new carbohydrate-specific modification strategy results in minimal loss of higher order protein structure after modification, and thus maintains the three dimensional conformation necessary for optimal vaccine efficacy.

### Example 9

### Terminal Galactose-Specific Modification of H1N1 Whole Virus Using a Combination of Galactose Oxidase and αGal Aminooxy Linker 32 (CAL-a11)

### Oxidation of H1N1 Virus by GO.

Egg derived PR8 H1N1 whole virus was modified by addition of an αGal aminooxy linker. The whole virus was inactivated by β-propiolactone (BPL) before modification. Ten microliters of catalase (10 U/μl) and 10 μl of GO (500 U/ml; SigmaG7907-150UN) were added to each 100 μl of inactivated virus (1 μg/μl; PR8 H1N1). After incubation at 37° C. for 2 hours, the mixture was ultra-centrifuged at 21000 g for 30 minutes to pellet the virus. The supernatant was discarded, and the pellet was resuspended in 200 μl 1×PBS, and ultra-centrifuged again. The supernatant was discarded, and pellet was resuspended with 150 μl 0.1 M NaOAc buffer.

### Conjugation

Ten microliters of αGal aminooxy linker (25 mg/mL) and 0.75 μl of aniline was added to the oxidized virus suspension from previous step. The reaction mixture was shaken overnight at 4° C., and then ultra-centrifuged at 21000 g for 30 minutes to pellet the virus. The supernatant was discarded, and the pellet was resuspended in 200 μl 1×PBS, and ultra-centrifuged again. The ultra-centrifugation was repeated two more times. The final pellet was resuspended in 100 μl of 1×PBS (containing 4% sucrose) and stored at −20° C.

Characterization of αGal-Virus Conjugate SDS-PAGE and western blot

FIG. 15 shows the (A) SDS-PAGE, (B) anti-HA western blot, and (C) anti-αGal western blot assays for this modification. Approximately 400 ng of HA1 protein was loaded in each lane. Lane 1 contains the original, unmodified inactivated virus sample, lanes 2 and 3 contain αGal aminooxy linker modified inactivated virus, and lane 4 contains the inactivated virus oxidized by GO only. Shifts of HA1 bands from lanes 2 and 3 on both the SDS-PAGE and anti-HA western blot indicate the successful modification of the virus with the αGal epitope. The anti-αGal western blot (C) further confirms that αGal is successfully installed on samples from lanes 2 and 3.

### Example 10

### Immobilization of Galactose Oxidase (iGO) Using NHS-Activated Agarose

Immobilization of galactose oxidase to agarose beads, serves the purpose of providing a way to separate the GO from the glycoprotein antigen after the initial step of glycoprotein oxidation. Seventy milligrams of dry NHS-Activated Agarose resin (Thermo Fisher Scientific Inc., IL) was added to an empty spin column (Bio-Rad, CA). One milliliter of galactose oxidase solution (30 U/mL) in 1×PBS was then added to the column containing dry resin. The top cap on the column was replaced and the reaction was mixed end-over-end for 1 hour. The top and bottom caps were removed and the column was placed in a collection tube. The column was centrifuged at 1000×g for 1 minute and flow-through was discarded. The resin was washed with 0.3 mL of 1×PBS two more times by centrifugation at 1000×g for 1 minute and all flow-through was discarded. 0.5 mL of 1 M Tris buffer (pH 8.0) was added to the column and the bottom and top caps were replaced. The column was mixed end-over-end for 15 minutes at room temperature. The top and bottom caps of the column were removed, and the column was then placed in a new collection tube, centrifuged at 1000×g for 1 minute and the flow-through was discarded. The column was washed with 0.3 mL 1×PBS two more times and all flow-through was discarded. For storage, 0.5 mL of 1×PBS was added to the column to result in 1 mL immobilized galactose oxidase suspension. The top and bottom caps were replaced and the column with final product was stored upright at 4° C.

### Example 11

### Terminal Galactose-Specific Modification of H1N1 Recombinant HA (rHA) Using a Combination of Immobilized Galactose Oxidase (i-GO) and αGal Aminooxy Linker 32 (CAL-a11)

Oxidation of H1N1 rHA by i-GO

Twenty microliters of neuraminidase (1 U/ml) and 100 μl of i-GO (30 U/ml) were added to 100 μl of rHA (0.66 mg/ml; Sino Biological Inc., China) in 1×PBS in a spin column. The top cap was replaced on the column. After incubation at 37° C. for 3 hours, the column was centrifuged at 1000×g for 2 minutes and the flow-through was collected. The resin was washed two more times using 1×PBS at 1000 ×g for 2 minutes each time, and all the flow-through was collected. The combined flow-through was ultra-centrifuged at 14,000×g using 10 kDa cut-off filter device (Millipore, MA) for 10 minutes and the flow-through was discard. The product was washed one more time by ultracentrifugation using 0.4 ml of 1 M NaOAc buffer (pH 5.5) at 14,000×g for 10 minutes. The final product was obtained as a 100 μl solution by adjusting the volume with 1 M NaOAc buffer (pH 5.5).

Conjugation with Linker 32 (CAL-a11)

Five microliters of αGal aminooxy linker (20 mg/mL) and 0.5 μL of aniline was added to 100 μl of oxidized rHA solution from previous step. The reaction mixture was shaken overnight at 4° C., and then ultra-centrifuged at 14,000×g using a 10 kDa cut-off filter device (Millipore, MA) for 10 minutes, and the flow-through was discarded. The ultra-centrifugation was repeated two more times using 1×PBS. The final product was obtained as a 100 μl solution by adjusting the volume with 1×PBS and was stored at −20° C.

### Characterization of αGal-rHA Conjugate

FIG. 16 shows the (A) SDS-PAGE, (B) anti-αGal western blot assays for this modification. Approximately 400 ng of HA protein was loaded in each lane. Lane 1 contains the original unmodified rHA sample, lane 2 contains the rHA treated with neuraminidase and i-GO, and lane 3 is the product after conjugation of the rHA with αGal aminooxy linker 32. The SDS-PAGE clearly indicates the successful addition of αGal onto rHA, since lane 3 shows significant shift compared to the migration pattern observed in lane 2. The anti-αGal western blot (B) further confirms that αGal linker 32 was successfully installed on the rHA protein.

### Example 12

### Terminal Galactose-Specific Modification of NA Co-Transfected H5N1 Recombinant HA (H5) Using a Combination of Immobilized Galactose Oxidase (i-GO) and αGal Aminooxy Linker

Oxidation of H1N1 H5 by i-GO

Four hundred microliters of i-GO (30 U/ml) was added to 100 μl of H5 (1.70 mg/ml) in 1×PBS in a spin column. The top cap was replaced on the column. After incubation at 37° C. for 4 hours, the column was centrifuged at 1000×g for 2 minutes and the flow-through was collected. The resin was washed two more times using 1×PBS at 1000 ×g for 2 minutes each time, and all the flow-through was collected. The combined flow-through was ultra-centrifuged at 14,000×g using 10 kDa cut-off filter device (Millipore, MA) for 10 minutes and the flow-through was discard. The product was washed one more time by ultracentrifugation using 0.4 ml of 1 M NaOAc buffer (pH 5.5) at 14,000×g for 10 minutes. The final product was obtained as a 600 μl solution by adjusting the volume with 1 M NaOAc buffer (pH 5.5).

Conjugation with Spacer Sp11

One microliter of sp11 (30 mg/mL) and 1.0 μL of aniline were added to 200 μl of oxidized H5 solution from previous step. The reaction mixture was shaken overnight at 4° C., and then ultra-centrifuged at 14,000×g using a 10 kDa cut-off filter device (Millipore, MA) for 10 minutes, and the flow-through was discarded. The ultra-centrifugation was repeated two more times using 1×PBS. The final product was obtained as a 100 μl solution by adjusting the volume with 1×PBS and was stored at −20° C.

Conjugation with Linker 32 (CAL-a11)

Four microliters of CAL-a11 (20 mg/mL) and 1.0 μL of aniline were added to 200 μl of oxidized H5 solution from previous step. The reaction mixture was shaken overnight at 4° C., and then ultra-centrifuged at 14,000×g using a 10 kDa cut-off filter device (Millipore, MA) for 10 minutes, and the flow-through was discarded. The ultra-centrifugation was repeated two more times using 1×PBS. The final product was obtained as a 100 μl solution by adjusting the volume with 1×PBS and was stored at −20° C.

Conjugation with Linker 36 (CAL-aN11)

Four microliters of CAL-aN11 (20 mg/mL) and 1.0 μL of aniline were added to 200 μl of oxidized H5 solution from previous step. The reaction mixture was shaken overnight at 4° C., and then ultra-centrifuged at 14,000×g using a 10 kDa cut-off filter device (Millipore, MA) for 10 minutes, and the flow-through was discarded. The ultra-centrifugation was repeated two more times using 1×PBS. The final product was obtained as a 100 μl solution by adjusting the volume with 1×PBS and was stored at −20° C.

### Characterization of Conjugates

FIG. 17 shows the (A) SDS-PAGE, (B) anti-αGal western blot assays for this modification. Approximately 400 ng of HA protein was loaded in each lane. Lane 1 contains the original unmodified H5 sample, lane 2 contains the H5 modified by sp11, and lane 3 and 4 are the products after conjugations of the H5 with αGal aminooxy linker CAL-a11 and CAL-aN11, respectively. The SDS-PAGE clearly indicates the successful addition of αGal linkers onto H5, since lanes 3 and 4 show significant shift compared to the migration pattern observed in lane 1. The anti-αGal western blot (B) further confirms that αGal was successfully installed on the H5 protein.

### Example 13

### Terminal Galactose-Specific Modification of NA Co-Transfected H7N9 Recombinant HA (H7) Using a Combination of Immobilized Galactose Oxidase (i-GO) and αGal Aminooxy Linkers

Oxidation of H7N9 H7 by i-GO

Four hundred microliters of i-GO (30 U/ml) was added to 150 μl of H7 (1.0 mg/ml) in 1×PBS in a spin column. The top cap was replaced on the column. After incubation at 37° C. for 4 hours, the column was centrifuged at 1000×g for 2 minutes and the flow-through was collected. The resin was washed two more times using 1×PBS at 1000 ×g for 2 minutes each time, and all the flow-through was collected. The combined flow-through was ultra-centrifuged at 14,000×g using 10 kDa cut-off filter device (Millipore, MA) for 10 minutes and the flow-through was discard. The product was washed one more time by ultracentrifugation using 0.4 ml of 1 M NaOAc buffer (pH 5.5) at 14,000×g for 10 minutes. The final product was obtained as a 600 μl solution by adjusting the volume with 1 M NaOAc buffer (pH 5.5).

Conjugation with spacer sp11

One microliter of sp11 (30 mg/mL) and 1.0 μL of aniline were added to 200 μl of oxidized H7 solution from previous step. The reaction mixture was shaken overnight at 4° C., and then ultra-centrifuged at 14,000×g using a 10 kDa cut-off filter device (Millipore, MA) for 10 minutes, and the flow-through was discarded. The ultra-centrifugation was repeated two more times using 1×PBS. The final product was obtained as a 100 μl solution by adjusting the volume with 1×PBS and was stored at −20° C.

Conjugation with Linker 32 (CAL-a11)

Four microliters of CAL-a11 (20 mg/mL) and 1.0 μL of aniline were added to 200 μl of oxidized H7 solution from previous step. The reaction mixture was shaken overnight at 4° C., and then ultra-centrifuged at 14,000×g using a 10 kDa cut-off filter device (Millipore, MA) for 10 minutes, and the flow-through was discarded. The ultra-centrifugation was repeated two more times using 1×PBS. The final product was obtained as a 100 μl solution by adjusting the volume with 1×PBS and was stored at −20° C.

Conjugation with Linker 36 (CAL-aN11)

Four microliters of CAL-aN11 (20 mg/mL) and 1.0 μL of aniline were added to 200 μl of oxidized H7 solution from previous step. The reaction mixture was shaken overnight at 4° C., and then ultra-centrifuged at 14,000×g using a 10 kDa cut-off filter device (Millipore, MA) for 10 minutes, and the flow-through was discarded. The ultra-centrifugation was repeated two more times using 1×PBS. The final product was obtained as a 100 μl solution by adjusting the volume with 1×PBS and was stored at −20° C.

### Characterization of Conjugates

FIG. 18 shows the (A) SDS-PAGE, (B) anti-αGal western blot assays for this modification. Approximately 400 ng of HA protein was loaded in each lane. Lane 1 contains the original unmodified H7 sample, lane 2 contains the H7 modified by sp11, and lane 3 and 4 are the products after conjugations of the H7 with αGal aminooxy linker CAL-a11 and CAL-aN11, respectively. The SDS-PAGE clearly indicates the successful addition of spacer and αGal linkers onto H7, since lanes 2, 3 and 4 show significant shift compared to the migration pattern observed in lane 1. The anti-αGal western blot (B) further confirms that αGal was successfully installed on the H7 protein.

### Example 14

### Antibody Induction with Linker Modified VLPs

FIG. 19A shows the measurement of serum antibodies produced against hemagglutinin in mice vaccinated with either unmodified influenza VLPs, influenza VLPs modified with αGal- at carbohydrates (CAL-a11) or influenza VLPs modified with αGal at lysine residues (CAL-a04). FIG. 19B shows the structure of the CAL-a11 and CAL-a04 linkers.

To test the ability of αGal linker modified VLPs to induce an immune response against the immunizing antigen, αGT knockout mice were primed using pig kidney membrane extracts and CpG oligonucleotides in incomplete Freund's adjuvant which induced anti-αGal antibodies. Virus-like particles were made by transfecting 293F cells (which are αGal negative) with plasmids coding for H1 hemagglutinin (HA), N1 neuraminidase and M1 matrix protein from the Puerto Rico strain of influenza. The VLPs were purified by repeated centrifugation and polyethylene glycol precipitation. The VLPs were chemically modified with galactose oxidase to produce oxidizing carbohydrates, which was followed by linkage with the CAL-a11 linker (αGal addition to carbohydrates) or using the CAL-a04 linker N-hydroxysuccinimide-activated (αGal addition to lysine residues). Two weeks after their last priming with pig kidney membrane extracts and CpG oligonucleotides in incomplete Freund's adjuvant, mice were injected with VLPs containing 100 ng of HA protein. Five weeks later, the mice received a second VLP vaccination and two weeks later, blood was drawn. Serial dilutions of sera were tested by ELISA for antibody reactivity against recombinant, monomeric HA protein. The OD value of a 1:200 dilution of sera is presented here. As shown in FIG. 16, there is a highly significant difference in the serum OD values of mice injected with VLPs modified with the carbohydrate specific CAL-a11 linker compared to mice injected with unmodified VLPs (p=0.0105). There is also a significant difference in the OD values of the mice injected with VLPs modified with the CAL-a11 linker compared to those injected with VLPs modified with the lysine specific CAL-a04 linker (p=0.045). There is no statistical difference in the OD values of mice injected with unmodified VLPs and those injected with the lysine specific CAL-a04 linker. These data indicate that carbohydrate-specific modification of VLPs induced a strong antibody response against the unmodified glycoprotein antigen that was not observed when lysine modification of the VLPs was utilized.

### Example 15

### Immunization with αGal-Linker Modified Influenza Hemagglutinin (HA) Conjugates

The following immunizations are performed to induce immunity against influenza virus using αGal modification of the recombinant HA with the carbohydrate-specific linker chemistry. αGT knockout mice (of the BALB/c genetic background, H-2^{d}) are primed with pig kidney membrane extract with CpG DNA in incomplete Freund's adjuvant to induce anti-αGal antibodies. Additionally, wild type BALB/c mice, which do not develop anti-αGal antibodies are used as control groups. Each animal is immunized with two doses of 250 or 100 ng of purified influenza HA protein resuspended in a buffered saline solution, either with or without αGal. These experiments can be carried out with or without adjuvant. Examples of treatment and control groups and doses are:

| G# | Strain | Influenza Vaccine | Dose |
|---|---|---|---|
| 1 | αGT KO | none | — |
| 2 | αGT KO | αGal⁽⁻⁾ - rHA vaccine | 100 ng |
| 3 | αGT KO | αGal⁽⁻⁾ - rHA vaccine | 250 ng |
| 4 | αGT KO | αGal⁽⁺⁾ - rHA vaccine | 100 ng |
| 5 | αGT KO | αGal⁽⁺⁾ - rHA vaccine | 250 ng |
| 6 | BALB/c | none | — |
| 7 | BALB/c | αGal⁽⁻⁾ - rHA vaccine | 100 ng |
| 8 | BALB/c | αGal⁽⁻⁾ - rHA vaccine | 250 ng |
| 9 | BALB/c | αGal⁽⁺⁾ - rHA vaccine | 100 ng |
| 10 | BALB/c | αGal⁽⁺⁾ - rHA vaccine | 250 ng |

The vaccines are administered by subcutaneous or intradermal injection, and each dose is administered two to four weeks apart. Challenge with virus is performed two to four weeks after the last vaccination. Immunologic tests are conducted one week after the last immunization as described below.

It has been previously shown that αGal-positive vaccines induce varied immune responses that are specific to the modified vaccine (Abdel-Motal, et al., 2006). Mice given unmodified influenza vaccine (with adjuvant) have greatly enhanced protection from lethal influenza challenge. As demonstrated in Abdel-Motal et al. (2006), 90% of mice vaccinated with heat-killed egg-derived influenza virus without αGal died when challenged with influenza virus. However, when mice were vaccinated with heat-killed egg-derived influenza virus with αGal, only 10% of mice died when challenged with influenza. The presence of αGal epitopes elicits the formation of immunocomplexes, which are able to elicit an immune response even in the absence of adjuvant. Analysis of the immune response parameters obtained after the immunization treatments described above provide information regarding the effect of the αGal epitope on the immunogenicity of recombinant protein vaccine, the effects of the αGal epitope on the potency or dose necessary to achieve certain levels of immune response, the effect of the presence of anti-αGal antibodies on the final immune response and the numbers of αGal epitopes per molecule that produce the highest immune protection.

### Example 16

### Evaluation of Immune Response in Mice after Vaccination with αGal Modified Recombinant HIM HA Conjugates

After immunization with recombinant influenza vaccine, there will be a significant enhancement in immune parameters when the immunizing antigen is αGal⁽⁺⁾ relative to when the immunizing antigen is αGal⁽⁻⁾. Mice vaccinated with αGal⁽⁺⁾ and αGal⁽⁻⁾ vaccines are bled and the serum antibody titers to influenza proteins are tested. Specific immunoglobulin (Ig) classes are tested in order to determine which type of immunoglobulin is predominant in this vaccination scenario.

In addition to B cell and antibody responses, splenocytes from mice vaccinated with αGal⁽⁺⁾ or αGal⁽⁻⁾ recombinant influenza protein vaccines are harvested and cultured for 6 hours in the presence or absence of stimulation. The control for maximum stimulation is the ionophore PMA/Ca⁺⁺. 10⁶ splenocytes are cultured with dendritic cells isolated from BALB/c mice. These cultures are either unstimulated (no exogenous antigen added) or given influenza protein (heat-killed virus). After incubation, cells are harvested and cultured on 96-well filter plates and the filters are developed for antibody staining for IFNγ and/or TNFα in ELISPOT. The number of spots detected as a function of the number of splenocytes added to the well is determined. Alternatively, after incubation cells are harvested and stained for intracellular IFNγ and/or TNFα. Detection is performed by FACS gating for lymphocytes in the forward scatter plot. The percentage of lymphocytes activated by PMA/Ca++ ionophore is considered the maximum activation detectable in this experiment. Resting (unstimulated) T cells and T cells stimulated with influenza proteins have undetectable intracellular IFNγ or TNF-α, indicating that no T cells precursors are able to recognize influenza antigens without prior stimulation, while vaccination with αGal⁽⁻⁾ vaccine gives only modest T cell stimulation. On the contrary, vaccination with αGal⁽⁺⁾ influenza vaccine induces T cell precursors that specifically recognize influenza proteins in vitro. Additionally, the number of precursors in spleens from mice vaccinated with αGal⁽⁺⁾ vaccine is superior relative to the number of precursors observed in spleens of mice vaccinated with αGal⁽⁻⁾ influenza vaccine. This results indicate that these T cells induced after vaccination with αGal⁽⁺⁾ recombinant influenza vaccine are responsible for enhanced immunity in mice challenged with lethal influenza virus.

In a different set of experiments, cell-surface activation markers are used to measure specific T cell recognition of the αGal⁽⁻⁾ influenza vaccine. It is well described that upon engagement of the T cell receptor (TCR), T cells up-regulate several cell surface molecules that indicate an activated state of the lymphocyte. One of those molecules is the IL-2 receptor a chain or CD25. Upon TCR engagement, CD25 is up-regulated and can be detected by FACS at 1 day after activation. Similarly, CD69 (or very early activation antigen (VEA)) is up-regulated upon T cell activation. CD69 functions as a signal-transmitting receptor in different cells, it is involved in early events of lymphocyte activation and contributes to T cell activation by inducing synthesis of different cytokines, and their receptors. Both activation markers (CD25 and CD69) are expressed at very low level in resting T cells. To demonstrate that vaccination with αGal⁽⁺⁾ recombinant influenza proteins induced T cell precursors able to recognize specifically influenza, the up-regulation of activation markers is used as parameters to measure recognition and activation. Cells are harvested from the spleens of mice vaccinated with αGal⁽⁻⁾ or αGal⁽⁺⁾ influenza proteins. These cells are cultured without stimulation or stimulated with αGal⁽⁻⁾ influenza proteins. After 24 hours of culture, cell are harvested and stained to detect CD25 or CD69 by FACS. Resting T cells (no stimulation) and cells from mice vaccinated with αGal(−) influenza vaccine show very low levels of activated CD25(+) and CD69(+) lymphocytes. On the other hand, increased numbers of activated (CD25⁽⁺⁾ and CD69⁽⁺⁾) lymphocytes from mice vaccinated with αGal⁽⁺⁾ influenza protein are seen when T cells are cultured with αGal⁽⁻⁾ influenza proteins.

### Example 17

### Immunization with αGal-Modified Virus-Like Particle (VLPs) Vaccines

The following immunizations are performed with VLPs using αGal modification of the VLPs with the carbohydrate-specific linker chemistry. αGT knockout mice (of the BALB/c genetic background, H-2^{d}) are primed with pig kidney membrane extract with CpG DNA in incomplete Freund's adjuvant to induce anti-αGal antibodies. Additionally, wild type BALB/c mice, which do not develop anti-αGal antibodies are used as control groups. Each animal is immunized with two doses of 250 or 100 ng of VLPs resuspended in a buffered saline solution, either with or without αGal. These experiments can be carried out with or without adjuvant. Examples of possible treatment and control groups and doses are:

| G# | Strain | VLP Vaccine | Dose |
|---|---|---|---|
| 1 | αGT KO | none | — |
| 2 | αGT KO | αGal⁽⁻⁾ - Virus-like particle vaccine | 100 ng |
| 3 | αGT KO | αGal⁽⁻⁾ - Virus-like particle vaccine | 250 ng |
| 4 | αGT KO | αGal⁽⁺⁾ -Virus-like particle vaccine | 100 ng |
| 5 | αGT KO | αGal⁽⁺⁾ - Virus-like particle vaccine | 250 ng |
| 6 | BALB/c | none | — |
| 7 | BALB/c | αGal⁽⁻⁾ - Virus-like particle vaccine | 100 ng |
| 8 | BALB/c | αGal⁽⁻⁾ -Virus-like particle vaccine | 250 ng |
| 9 | BALB/c | αGal⁽⁺⁾ - Virus-like particle vaccine | 100 ng |
| 10 | BALB/c | αGal⁽⁺⁾ - Virus-like particle vaccine | 250 ng |

The vaccines are administered by subcutaneous or intradermal injection, and each dose is administered two to four weeks apart. Challenge with virus is performed two to four weeks after the last vaccination. Immunologic tests are conducted one week after the last immunization as described below.

The vaccines are administered by subcutaneous or intradermal injection, and each dose is administered two to four weeks apart. Challenge with virus is performed two to four weeks after the last vaccination. Immunologic tests are conducted one week after the last immunization as described below. VLPs are a unique type of vaccinating molecule. When virus proteins are assembled into a VLP, the structure resembles that of the virus from which the proteins were derived, such that the particle can “infect” a cell (Roldão et al., 2010). Given the fact that these particles bind to cells using viral surface proteins, those proteins can subsequently be processed in a manner similar to when viruses infect cells. This means that viral proteins delivered using VLP vaccines can be processed intracellularly using the MHC class I machinery. This unique trait means that viral antigens encoded by VLPs are processed differently than proteins given in typical vaccines. The VLP is created by transfecting or transducing a cell with genes for key influenza proteins (such as hemagglutinin (HA), neuraminidase (NA), matrix protein-1 (M1) and/or matrix protein-2 (M2)). The VLPs are denser than other extracellular material and can thus be precipitated using high speed centrifugation and/or tangential flow filtration (TFF). Additional purification steps give material that under electron microscopy resembles influenza virions. The vaccine is quantitated by measuring the HA content in a given vaccine preparation (for instance, one dose would be 250 ng of HA in the VLP). The VLP is then modified with carbohydrate linker to make it αGal⁽⁺⁾. The vaccine is diluted in a buffered saline solution and delivered via subcutaneous or intradermal routes. Mice are subsequently challenged with influenza virus in order to determine the protective efficacy of the vaccines.

### Example 18

### Evaluation of Immune Response in Mice after Vaccination with αGal Modified Virus-Like Particle Vaccines

After immunization with VLP vaccine, there is a significant enhancement in immune parameters when the immunizing VLP is αGal⁽⁺⁾ relative to when the immunizing VLP is αGal⁻. Mice vaccinated with αGal⁽⁺⁾ and αGal⁽⁻⁾ VLPs are bled and the serum antibody titers to influenza proteins are tested. Specific immunoglobulin (Ig) classes are tested in order to determine which type of Ig is predominant in this vaccination scenario. In addition to B cell and antibody responses, splenocytes from mice vaccinated with αGal⁽⁺⁾ or αGal⁽⁻⁾ VLP vaccines are harvested and cultured for 6 hours in the presence or absence of stimulation. The control for maximum stimulation is the ionophore PMA/Ca⁺⁺. 10⁶ splenocytes are cultured with dendritic cells isolated from BALB/c mice. These cultures are either unstimulated (no exogenous antigen added) or given influenza protein (heat-killed virus). After incubation, cells are harvested and cultured on 96-well filter plates and the filters are developed for antibody staining for IFNγ and/or TNFα in ELISPOT. The number of spots detected as a function of the number of splenocytes added to the well is determined. Alternatively, after incubation cells are harvested and stained for intracellular IFNγ and/or TNFα. Detection is performed by FACS gating for lymphocytes in the forward scatter plot. The percentage of lymphocytes activated by PMA/Ca++ ionophore is considered the maximum activation detectable in this experiment. Resting (unstimulated) T cells and T cells stimulated with influenza proteins have undetectable intracellular IFNγ or TNF-α, indicating that no T cells precursors are able to recognize influenza antigens without prior stimulation, while vaccination with αGal⁽⁻⁾ VLP gives only modest T cell stimulation. On the contrary, vaccination with αGal⁽⁺⁾ influenza VLP induces T cell precursors that specifically recognize influenza proteins in vitro. Additionally, the number of precursors in spleens from mice vaccinated with αGal⁽⁺⁾ VLPs is expected to be superior relative to the number of precursors observed in spleens of mice vaccinated with αGal⁽⁻⁾ influenza VLPs. This result indicates that these T cells induced after vaccination with αGal⁽⁺⁾ VLPs are responsible for enhanced immunity in mice challenged with lethal influenza virus.

In a different set of experiments, cell-surface activation markers are used to measure specific T cell recognition of the αGal⁽⁻⁾ influenza VLPs. Cells are harvested from the spleens of mice vaccinated with αGal⁽⁻⁾ or αGal⁽⁺⁾ VLP vaccines. These cells are cultured without stimulation or stimulated with αGal⁽⁻⁾ influenza proteins. After 24 hours of culture, cell are harvested and stained to detect CD25 or CD69 by FACS. Resting T cells (no stimulation) and cells from mice vaccinated with αGal(−) influenza vaccine show very low levels of activated CD25(+) and CD69(+) lymphocytes. On the other hand, increased numbers of activated (CD25⁽⁺⁾ and CD69⁽⁺⁾) lymphocytes arise in from mice vaccinated with αGal⁽⁺⁾ influenza VLPs when T cells are cultured with αGal⁽⁻⁾ influenza proteins.

### Example 19

### Evaluation of Antibody Response in Mice after Vaccination with αGal Modified H1N1 Virus-Like Particle Vaccines

FIG. 20 shows the antibody response after immunization of mice with H1N1 influenza virus-like particles (VLPs) modified with CAL-a11 αGal linker, compared to the antibody responses induced by control VLPs. The hemagglutinin protein (HA) content of both control VLPs and CAL-a11-modified VLPs were quantitated and VLPs containing a total of 100 ng of HA protein were injected subcutaneously into mice twice, four weeks apart. Two weeks after the second injection, blood was drawn and serum collected. The level of antibody against H1-HA protein was examined using ELISA. Each point in the graph represents an individual mouse. Statistical analysis was conducted between groups using unpaired t-Test (two-tailed). These data demonstrate that there is a highly significant increase in antibody titer when the candidate VLP vaccine is modified with the αGal linker.

### Example 20

### Evaluation of Antibody Response in Mice after Vaccination with αGal Modified H5N1 Virus-Like Particle Vaccines

FIG. 21 shows the antibody response after immunization of mice with H5N1 influenza recombinant protein vaccine modified with CAL-a11 αGal linker, compared to the antibody responses induced by unmodified or spacer only modified control VLPs. H5N1 trimeric vaccines induce a higher antibody response when modified with CAL-a11 αGal linker. An H5 recombinant protein vaccine was made in 293F cells. A gene construct with the H5 protein gene was fused to a heterologous signal sequence. At the 3′ end, sequences were added coding for a trimerization domain and a poly-histidine tag. The construct was transfected into 293F cells and supernatant collected. The protein was purified by affinity chromatography and quantified. The protein was either not modified (rHA5), modified with a linker containing all components of the CAL-a11 linker except for the αGal trisaccharide (rHA5+SP11) or modified with the CAL-a11 linker (rHA5+CAL-a11). A total of 100 ng of HA protein was injected subcutaneously into mice twice, four weeks apart, in phosphate-buffered saline in the absence of adjuvant. Two weeks after the last injection, blood was drawn and serum collected. The level of antibody against H5-HA protein (not the αGal-modified form) was examined using ELISA. Each point in the graph represents an individual mouse at a serum dilution of 1:400. Statistical analysis was examined between groups using unpaired t-Test (two-tailed). These data demonstrate that there is a highly significant increase in antibody titer when the candidate H5 vaccine is modified with the αGal linker and that the specific portion of the linker responsible for the increased titer is the αGal trisaccharide.

### Example 21

### Evaluation of Antibody Response in Mice after Vaccination with αGal Modified H7N9 Trimeric Vaccines

FIG. 22 shows the antibody response after immunization of mice with H7N9 trimeric vaccines. H7N9 trimeric vaccines induce a higher antibody response when modified with CAL-a11 linker and gives and even higher response when the trisaccharide contains a proximal N-acetylglucosamine instead of glucose (CAL-aN11). An H7 recombinant protein vaccine was made in 293F cells. A gene construct with the H7 protein gene was fused to a heterologous signal sequence. At the 3′ end, sequences were added coding for a trimerization domain and a poly-histidine tag. The construct was transfected into 293F cells and supernatant collected. The protein was purified by affinity chromatography and quantified. The protein was either not modified (rHA7), modified with a linker containing all components of CAL-a11 except for the αGal trisaccharide (rHA7 SP11), modified linker containing the trisaccharide with glucose at the reducing end (rHA7 CAL-a11) or modified with linker containing N-acetylglucosamine at the reducing end (rHA7 CAL-aN11). A total of 100 ng of HA protein was injected subcutaneously into mice twice, four weeks apart. Two weeks after the last injection, blood was drawn and serum collected. The level of antibody against H7 protein (not the αGal-modified form) was examined using ELISA. Each point in the graph represents an individual mouse. Statistical analysis was conducted between groups using unpaired t-Test (two-tailed). These data demonstrate that modification of H7 pandemic influenza vaccine with αGal-containing linker molecules results in a significantly higher antibody levels against H7 HA protein.

### Example 22

### Enhancement of Survival Elicited by Vaccination with αGal Modified Virus-Like Particle Vaccines after a Lethal Challenge with Flu Virus

FIG. 23 shows the enhancement in survival and protection after a lethal challenge of mice with H1N1 influenza virus. H1N1 virus-like particles (VLPs) modified with CAL-a11 αGal linker protect mice from influenza mortality. The HA content of both control VLPs and CAL-a11-modified VLPs were quantitated by Western blot against appropriate standards and VLPs containing a total of 100 ng of HA protein in phosphate-buffered saline without any adjuvant were injected subcutaneously into mice twice, four weeks apart Two to four weeks after the second vaccination, the mice were challenged with a lethal dose (10×LD₅₀) of the H1N1 A/Puerto Rico/8/34 mouse-adapted influenza virus by intranasal instillation. Mice were examined daily for health and weight loss and animals sacrificed if weight loss approached 30% or if they were overtly moribund. Data are presented as percent survival at the indicated days post-infection. Statistical analysis was conducted between groups using log-rank (Mantel-Cox) test. These data demonstrate when vaccinated with unmodified VLPs, only 50% of the mice survive challenge while 90% of mice vaccinated with αGal linker-modified VLPs survive. This is highly significant increase in survival.

### Example 23

### Immunization with αGal Modified Whole Viral Vaccine Conjugates

The following immunizations are performed with whole virus inactivated vaccine using αGal modification of the VLPs with the carbohydrate-specific linker chemistry. αGT knockout mice (of the BALB/c genetic background, H-2^{d}) are primed with pig kidney membrane extract with CpG DNA in incomplete Freund's adjuvant to induce anti-αGal antibodies. Additionally, wild type BALB/c mice, which do not develop anti-αGal antibodies are used as control groups. Each animal is immunized with two doses of 250 or 100 ng of whole virus vaccine resuspended in a buffered saline solution, either with or without αGal. These experiments can be carried out with or without adjuvant. Examples of treatment and control groups and doses are:

| G# | Strain | Whole virus vaccine | Dose |
|---|---|---|---|
| 1 | αGT KO | none | — |
| 2 | αGT KO | αGal⁽⁻⁾ - heat-inactivated viral vaccine | 100 ng |
| 3 | αGT KO | αGal⁽⁻⁾ - heat-inactivated viral vaccine | 250 ng |
| 4 | αGT KO | αGal⁽⁺⁾ - heat-inactivated viral vaccine | 100 ng |
| 5 | αGT KO | αGal⁽⁺⁾ - heat-inactivated viral vaccine | 250 ng |
| 6 | BALB/c | none | — |
| 7 | BALB/c | αGal⁽⁻⁾ - heat-inactivated viral vaccine | 100 ng |
| 8 | BALB/c | αGal⁽⁻⁾ - heat-inactivated viral vaccine | 250 ng |
| 9 | BALB/c | αGal⁽⁺⁾ - heat-inactivated viral vaccine | 100 ng |
| 10 | BALB/c | αGal⁽⁺⁾ - heat-inactivated viral vaccine | 250 ng |

The vaccines are administered by subcutaneous or intradermal injection, and each dose is administered two to four weeks apart. Challenge with virus is performed two to four weeks after the last vaccination. Immunologic tests are conducted one week after the last immunization as described below.

One issue with vaccines using recombinant subunits or VLPs is that the other proteins that make up the influenza virus are not in the vaccine and thus do not contribute to the resulting immune response. Whole virus inactivated vaccines make use of the entire array of viral proteins in order to make a more complete vaccine (Dormitzer et al, 2012). The virus is inactivated by chemical means such as formalin or beta-propriolactone and the preparation is purified. The vaccine is quantitated by measuring the HA content in a given vaccine preparation (for instance, one dose would be 250 ng of HA in the VLP). The whole virus vaccine is then modified with carbohydrate linker to make it αGal⁽⁺⁾. The vaccine is diluted in a buffered saline solution and delivered via subcutaneous or intradermal routes. Mice are subsequently challenged with influenza virus in order to determine the protective efficacy of the vaccines.

### Example 24

### Evaluation of Immune Response in Mice after Vaccination with αGal-Modified Whole Viral Vaccine Conjugates

It is expected that after immunization with whole virus influenza vaccine, there will be a significant enhancement in immune parameters when the immunizing vaccine is αGal⁽⁺⁾ relative to when the immunizing whole virus vaccine is αGal⁽⁻⁾. Mice vaccinated with αGal⁽⁺⁾ and αGal⁽⁻⁾ whole virus are bled and the serum antibody titers to influenza proteins are tested. Specific immunoglobulin (Ig) classes are tested in order to determine which type of Ig is predominant in this vaccination scenario. In addition to B cell and antibody responses, splenocytes from mice vaccinated with αGal⁽⁺⁾ or αGal⁽⁻⁾ whole virus vaccines are harvested and cultured for 6 hours in the presence or absence of stimulation. The control for maximum stimulation is the ionophore PMA/Ca⁺⁺. 10⁶ splenocytes are cultured with dendritic cells isolated from BALB/c mice. These cultures are either unstimulated (no exogenous antigen added) or given influenza protein (heat-killed virus). After incubation, cells are harvested and cultured on 96-well filter plates and the filters are developed for antibody staining for IFNγ and/or TNFα in ELISPOT. The number of spots detected as a function of the number of splenocytes added to the well is determined. Alternatively, after incubation cells are harvested and stained for intracellular IFNγ and/or TNFα. Detection is performed by FACS gating for lymphocytes in the forward scatter plot. The percentage of lymphocytes activated by PMA/Ca++ ionophore is considered the maximum activation detectable in this experiment. Resting (unstimulated) T cells and T cells stimulated with influenza proteins have undetectable intracellular IFNγ or TNF-α, indicating that no T cells precursors are able to recognize influenza antigens without prior stimulation, while vaccination with αGal⁽⁻⁾ whole virus give only modest T cell stimulation. To the contrary, vaccination with αGal⁽⁺⁾ influenza whole virus vaccine induce T cell precursors that specifically recognize influenza proteins in vitro. Additionally, the number of precursors in spleens from mice vaccinated with αGal⁽⁺⁾ whole virus preparations is superior relative to the number of precursors observed in spleens of mice vaccinated with αGal⁽⁻⁾ influenza whole virus vaccine. This result suggest that these T cells induced after vaccination with αGal⁽⁺⁾ whole virus are responsible for enhanced immunity in mice challenged with lethal influenza virus.

In a different set of experiments, cell-surface activation markers can be used to measure specific T cell recognition of the αGal⁽⁻⁾ influenza whole virus vaccines To demonstrate that vaccination with αGal⁽⁺⁾ VLPs induced T cell precursors able to recognize specifically influenza, the up-regulation of activation markers can be used as parameters to measure recognition and activation. Cells are harvested from the spleens of mice vaccinated with αGal⁽⁻⁾ or αGal⁽⁺⁾ whole virus vaccines. These cells are cultured without stimulation or stimulated with αGal⁽⁻⁾ influenza proteins. After 24 hours of culture, cell are harvested and stained to detect CD25 or CD69 by FACS. Resting T cells (no stimulation) and cells from mice vaccinated with αGal(−) influenza vaccine show very low levels of activated CD25(+) and CD69(+) lymphocytes. On the other hand, increased numbers of activated (CD25⁽⁺⁾ and CD69⁽⁺⁾) lymphocytes from mice vaccinated with αGal⁽⁺⁾ influenza whole virus vaccine are seen when T cells are cultured with αGal⁽⁻⁾ influenza proteins.

While specific embodiments of the invention have been described and illustrated, such embodiments should be considered illustrative of the invention only and not as limiting the invention as construed in accordance with the accompanying claims.

All patents, applications, and other references cited herein are incorporated by reference in their entireties.

### REFERENCES

1. Agnihotri, G.; Tiwari, P.; Misra, A. K., One-pot synthesis of per-O-acetylated thioglycosides from unprotected reducing sugars. Carbohydr Res 2005, 340 (7), 1393-6.
2. Hsieh, S. Y.; Jan, M. D.; Patkar, L. N.; Chen, C. T.; Lin, C. C., Synthesis of a carboxyl linker containing Pk trisaccharide. Carbohydr Res 2005, 340 (1), 49-57.
3. Xue, J.; Zhu, J.; Marchant, R. E.; Guo, Z., Pentaerythritol as the core of multivalent glycolipids: synthesis of a glycolipid with three SO3Lea ligands. Org Lett 2005, 7 (17), 3753-6.
4. Mandal, P. K.; Misra, A. K., Mild and Efficient Hydrolysis of Thioglycosides to Glycosyl Hemiacetals Using N-Iodosaccharin. Synlett 2007, 8, 1207-1210.
5. Nagorny, P.; Fasching, B.; Li, X.; Chen, G.; Aussedat, B.; Danishefsky, S. J., Toward fully synthetic homogeneous beta-human follicle-stimulating hormone (beta-hFSH) with a biantennary N-linked dodecasaccharide. synthesis of beta-hFSH with chitobiose units at the natural linkage sites. J Am Chem Soc 2009, 131 (16), 5792-9.
6. Bennett, C. S.; Dean, S. M.; Payne, R. J.; Ficht, S.; Brik, A.; Wong, C. H., Sugar-assisted glycopeptide ligation with complex oligosaccharides: scope and limitations. J Am Chem Soc 2008, 130 (36), 11945-52.
7. Arranz-Plaza, E.; Tracy, A. S.; Siriwardena, A.; Pierce, J. M.; Boons, G. J., High-avidity, low-affinity multivalent interactions and the block to polyspermy in Xenopus laevis. J Am Chem Soc 2002, 124 (44), 13035-46.
8. Foillard, S.; Rasmussen, M. O.; Razkin, J.; Boturyn, D.; Dumy, P., 1-Ethoxyethylidene, a new group for the stepwise SPPS of aminooxyacetic acid containing peptides. J Org Chem 2008, 73 (3), 983-91.
9. Mao, L.; Wang, H.; Tan, M.; Ou, L.; Kong, D.; Yang, Z., Conjugation of two complementary anti-cancer drugs confers molecular hydrogels as a co-delivery system. Chem Commun (Camb) 2012, 48 (3), 395-7.
10. Maley, F.; Trimble, R. B.; Tarentino, A. L.; Plummer, T. H., Jr., Characterization of glycoproteins and their associated oligosaccharides through the use of endoglycosidases. Anal Biochem 1989, 180 (2), 195-204; (b) Plummer, T. H., Jr.; Tarentino, A. L., Purification of the oligosaccharide-cleaving enzymes of Flavobacterium meningosepticum. Glycobiology 1991, 1 (3), 257-63.
11. Robbins, P. W.; Trimble, R. B.; Wirth, D. F.; Hering, C.; Maley, F.; Maley, G. F.; Das, R.; Gibson, B. W.; Royal, N.; Biemann, K., Primary structure of the Streptomyces enzyme endo-beta-N-acetylglucosaminidase H. J Biol Chem 1984, 259 (12), 7577-83.
12. Wei C J, Xu L, Kong W P, Shi W, Canis K, Stevens J, Yang Z Y, Dell A, Haslam S M, Wilson I A, Nabel G J. 2008. Comparative efficacy of neutralizing antibodies elicited by recombinant hemagglutinin proteins from avian H5N1 influenza virus. J Virol. 82:6200-8.
13. Welsh J P, Lu Y, He X S, Greenberg H B, Swartz J R. 2012. Cell-free production of trimeric influenza hemagglutinin head domain proteins as vaccine antigens. Biotechnol Bioeng. 109:2962-9.
14. Du L, Zhao G, Sun S, Zhang X, Zhou X, Guo Y, Li Y, Zhou Y, Jiang S. 2013. A critical HA1 neutralizing domain of H5N1 influenza in an optimal conformation induces strong cross-protection. PLoS One. 8:e53568.
15. Abdel-Motal, U., S. Wang, S. Lu, K. Wigglesworth, and U. Galili, 2006 Increased immunogenicity of human immunodeficiency virus gp120 engineered to express Galalpha1 3Galbeta1-4GlcNAc-R epitopes. J Virol. 80:6943-51.
16. Roldão A, Mellado M C, Castilho L R, Carrondo M J, Alves P M. 2010. Virus-like particles in vaccine development. Expert Rev Vaccines. 9:1149-76.
17. Dormitzer P R, Tsai T F, Del Giudice G. 2012. New technologies for influenza vaccines. Hum Vaccin Immunother. 8:45-58.
18. Wenlan Chen, Ph.D. Thesis, Ohio State University, 2010 (osu1280856149).
19. Berzofsky, J A. 1993. Epitope selection and design of synthetic vaccines. Molecular approaches to enhancing immunogenicity and cross-reactivity of engineered vaccines Ann NY Acad Sci 690:256-64.
20. Berzofsky, J A, J D Ahlers and I M Belyakov. 2001. Strategies for designing and optimizing new generation vaccines. Nat Rev Immunol 1(3):209-19.
21. Rosenberg, S A, J C Yang et al. 1998. Immunologic and therapeutic evaluation of a synthetic peptide vaccine for the treatment of patients with metastatic melanoma. Nat Med. 4(3):321-7.
22. Fong, L, Y. Hou et al. 2001. Altered peptide ligand vaccination with FLt3 ligand expanded dendritic cells for tumor immunotherapy. PNAS USA 98(15):8809-14.
23. Rivoltini, L., Y. Kawakami et al. 1995. Induction of tumor-reactive CTL from peripheral blood and tumor-infiltrating lymphocytes of melanoma patients by in vitro stimulation with an immunodominant peptide of the human melanoma antigen MART-1. J. Immunol 154(5):2257-65.
24. Zaremba, S., E Barzaga et al. 1997. Identification of an enhancer agonist cytotoxic T lymphocyte peptide from human carcinoembryonic antigen. Cancer Res. 57(20):4570-7.
25. Parmiani, G., C. Castelli, et al. 2002. Cancer immunotherapy with peptide-based vaccines: what have we achieved? Where are we going? J Natl Cancer Inst 94(11):805-18.
26. Ackerman, A L, C. Kyritsis, R. Tampe and P. Cresswell (2005). Access of soluble antigens to the endoplasmic reticulum can explain cross-presentation by dendritic cells. Nat Immunol 6(1):107-13.
27. Heath, W R, G. T. Belz et al. 2004. Cross-presentation, dendritic cell subsets, and the generation of immunity to cellular antigens. Immunol Rev 199:9-26.
28. Heath, W R. And F R Carbone. 2001. Cross-presentation, dendritic cells, tolerance and immunity. Anny Rev Immunol 19:47-64.
29. Palliser, D., E. Guillen, M. Ju, and H N Eisen. 2005. Multiple intracelluar routes in the cross-presentation of a soluble protein by murine dendritic cells. J. Immunol 174(4):1879-87.
30. Rafiq, K., A Bergtold, and R. Clynes. 2002. Immune complex-mediated antigen presentation induces tumor immunity. J. Clin Invest 110(1):71-9.
31. Schnurr, M. Q. Chen et al. 2005. Tumor antigen processing and presentation depend critically on dendritic cell type and the mode of antigen delivery. Blood 105(6):2465-72.
32. Benatuil, L., J. Kaye et al. 2005. The influence of natural antibody specificity on antigen immunogenicity. Eur J. Immunol 35(9):2638-47.
33. Galili, U. P M Repik, et al. 1996. Enhancement of antigen presentation of influenza virus hemagglutinin by the natural human anti-Gal antibody. Blood 82(8): 2485-93.
34. Janczuk et al. 2002. The synthesis of deoxy-α-Gal epitope derivatives for the evaluation of an anti-α-Gal antibody binding. Carbohydr Res 337(14):1247-59.
35. Wang et al, 1999. Enhanced inhibition of human anti-Gal antibody binding to mammalian cells by synthetic α-Gal epitope polymers. J. Am. Chem. Soc. 121(36):8174-8181.

We claim:

1. An immune adjuvant compound comprising a chemical structure Su-O—R₁—ONH₂, wherein Su is a monosaccharide, disaccharide, trisaccharide, tetrasaccharide or pentasaccharide, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups.

2. The immune adjuvant compound of claim 1, wherein Su is a αGal, Forssman, or L-Rhamnose epitope.

3. The immune adjuvant compound of claim 2, wherein αGal has the structure Gal(α1-3)Gal(β1-4)Glc or Gal(α1-3)Gal(β1-4)GlcNAc.

4. An isolated antigen comprising a modified glycoprotein wherein one or more carbohydrate residues in said glycoprotein have been chemically modified with an immune adjuvant compound comprising a chemical structure Su-O—R₁—ONH₂, wherein Su is a monosaccharide, disaccharide, trisaccharide, tetrasaccharide or pentasaccharide, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups.

5. The isolated antigen of claim 4, wherein Su is a αGal, Forssman, or L-Rhamnose epitope.

6. The isolated antigen of claim 5, wherein the αGal epitope has the structure Gal(α1-3)Gal(β1-4)Glc or Gal(α1-3)Gal(β1-4)GlcNAc.

7. The isolated antigen of claim 4, wherein said immune adjuvant compound is covalently bound to an oxidized carbohydrate residue present at a pre-existing N-linked or O-linked glycan in said glycoprotein.

8. The isolated antigen of claim 4, wherein said immune adjuvant compound does not alter the structure of said glycoprotein when bound.

9. The isolated antigen of claim 8 wherein said glycoprotein retains some or all of its natural biological activity.

10. The isolated antigen of claim 4, wherein said glycoprotein is a natural or synthetic polypeptide.

11. The isolated antigen of claim 4, wherein said glycoprotein is part of a VLP, a whole virus, or a whole cell.

12. The isolated antigen of claim 4 which elicits an immune response when administered to a subject.

13. The isolated antigen of claim 12 which elicits an immune response to an infectious agent or a tumor.

14. A pharmaceutical composition useful to elicit an immune response comprising an isolated antigen comprising a modified glycoprotein wherein one or more carbohydrate residues in said glycoprotein have been chemically modified with an immune adjuvant compound comprising a chemical structure Su-O—R₁—ONH₂, wherein Su is a monosaccharide, disaccharide, trisaccharide, tetrasaccharide or pentasaccharide, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups and a carrier.

15. The pharmaceutical composition of claim 14, wherein Su is a αGal, Forssman, or L-Rhamnose epitope.

16. The pharmaceutical composition of claim 15, wherein the αGal epitope has the structure Gal(α1-3)Gal(β1-4)Glc or Gal(α1-3)Gal(β1-4)GlcNAc.

17. The pharmaceutical composition of claim 14, wherein said immune adjuvant compound is covalently bound to an oxidized carbohydrate residue present at a pre-existing N-linked or O-linked glycan in said glycoprotein.

18. The pharmaceutical composition of claim 14, wherein said carbohydrate residue present at a pre-existing N-linked or O-linked glycan in the glycoprotein is a galactose residue.

19. The pharmaceutical composition of claim 14, wherein the oxidation of said carbohydrate residue present at a pre-existing N-linked or O-linked glycan in the glycoprotein is performed with galactose oxidase.

20. The pharmaceutical composition of claim 14, wherein said immune adjuvant compound does not alter the structure of said glycoprotein when bound.

21. The pharmaceutical composition of claim 14, wherein said glycoprotein retains some or all of its natural biological activity.

22. The pharmaceutical composition of claim 14, wherein said glycoprotein is a natural or synthetic polypeptide.

23. The pharmaceutical composition of claim 14, wherein said glycoprotein is part of a VLP, a whole virus, or a whole cell.

24. The pharmaceutical composition of claim 14 which elicits an immune response when administered to a subject.

25. The pharmaceutical composition of claim 24 which elicits an immune response to an infectious agent or a tumor when administered to a subject.

26. A method to induce an immune response in a subject against an antigen comprising administering to said subject an effective amount of an isolated antigen comprising a modified glycoprotein wherein one or more carbohydrate residues in said glycoprotein have been chemically modified with an immune adjuvant compound comprising a chemical structure Su-O—R₁—ONH₂, wherein Su is a monosaccharide, disaccharide, trisaccharide, tetrasaccharide or pentasaccharide, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups and a carrier.

27. The method of claim 26, wherein said subject is human.

28. The method of claim 26, wherein Su is a αGal, Forssman, or L-Rhamnose epitope.

29. The method of claim 28, wherein the αGal epitope has the structure Gal(α1-3)Gal(β1-4)Glc or Gal(α1-3)Gal(β1-4)GlcNAc.

30. The method of claim 26, wherein said immune adjuvant compound is covalently bound to an oxidized carbohydrate residues present at a pre-existing N-linked or O-linked glycan in said glycoprotein.

31. The method of claim 26, wherein said glycoprotein is a natural or synthetic polypeptide.

32. The method of claim 26, wherein said glycoprotein is part of a VLP, a whole virus, or a whole cell.

33. A method to produce an isolated antigen comprising a modified glycoprotein wherein one or more carbohydrate residues in said glycoprotein have been chemically modified with an immune adjuvant compound comprising a chemical structure Su-O—R₁—ONH₂, wherein Su is a monosaccharide, disaccharide, trisaccharide, tetrasaccharide or pentasaccharide, and wherein R1 is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups, by reacting said immune adjuvant compound with said glycoprotein to selectively attach said immune adjuvant compound to an oxidized carbohydrate residue present in said glycoprotein.

34. The method of claim 33, comprising the steps:
1) oxidizing a carbohydrate on said glycoprotein to produce a reactive carbonyl group, and
2) reacting said carbonyl group with the aminooxy group on said immune adjuvant compound to form an oxime bond and generate said isolated antigen.

35. The method of claim 34, wherein said oxidizing step is performed using an oxidant selected from the group consisting of NaIO4, galactose oxidase, or an engineered variant thereof.

36. The method of claim 35, wherein said galactose oxidase or engineered variant thereof is free or immobilized.

37. The method of claim 33, wherein said glycoprotein lacks a terminal galactose or N-acetylgalactosamine or sialic acid.

38. The method of claim 33, wherein said glycoprotein comprises an aldehyde group.

39. The isolated antigen produced by the method of claim 33.

40. An isolated antigen produced by a method comprising the steps of:
a) obtaining a vaccine preparation comprising a glycoprotein selected from the group of a purified glycoprotein or a glycoprotein that is part of a VLP, whole virus or cell
b) treating said vaccine preparation with an oxidizing agent selected from the group of NaIO4, galactose oxidase or an engineered variant thereof, to produce a reactive carbonyl group on one or more carbohydrate residues that form part of the glycan units of the glycoprotein
c) treating said oxidized vaccine preparation with an immune adjuvant compound of the structure Su-O—R1-ONH₂.
d) separating the oxidizing agent from the vaccine preparation.

41. The isolated antigen of claim 40, wherein Su is a αGal, Forssman, or L-Rhamnose epitope.

42. The isolated antigen of claim 41, wherein the αGal epitope has the structure Gal(α1-3)Gal(β1-4)Glc or Gal(α1-3)Gal(β1-4)GlcNAc.

43. The isolated antigen of claim 40, wherein said immune adjuvant compound is covalently bound to an oxidized carbohydrate residue present at a pre-existing N-linked or O-linked glycan in said glycoprotein.

44. The isolated antigen of claim 40, wherein said immune adjuvant compound does not alter the structure of said glycoprotein when bound.

45. The isolated antigen of claim 44 wherein said glycoprotein retains some or all of its natural biological activity.

46. The isolated antigen of claim 40 which elicits an immune response when administered to a subject.

47. The isolated antigen of claim 46 which elicits an immune response to an infectious agent or a tumor.

48. An isolated antigen comprising a modified glycoprotein having the structure Su-O—R₁—O—N═CR, wherein Su is a monosaccharide, disaccharide, trisaccharide, tetrasaccharide or pentasaccharide, and wherein CR represents the carbohydrate residue of said glycoprotein which is bound to N through an oxime bond, and wherein R₁ is any linear or branched alkyl group of 1 to 30 carbon atoms, wherein one or more carbon atoms in such alkyl group can be substituted by O, S, or N, and wherein one or more hydrogens can be substituted by hydroxyl, carbonyl, alkyl, sulphydryl or amino groups.

49. An isolated antigen comprising a modified glycoprotein having a saccharide epitope covalently bound at a carbohydrate residue present on said glycoprotein.

50. The isolated antigen of claim 49, wherein the saccharide epitope is a monosaccharide, disaccharide, trisaccharide, tetrasaccharide or pentasaccharide to which humans have natural pre-existing antibodies.

51. The isolated antigen of claim 49, wherein the saccharide epitope is bound to the carbohydrate residue via a linker.

52. The isolated antigen of claim 51, wherein the saccharide-linked glycoprotein has the structure Su-O—R₁—O—N=GP wherein R1 is any linear or branched alkyl group of 1 to 30 carbon atoms, wherein one or more carbon atoms in such alkyl group can be substituted by O, S, or N, wherein one or more hydrogens can be substituted by hydroxyl, carbonyl, alkyl, sulphydryl or amino groups, and wherein said N is double bonded to the carbohydrate residue of the glycoprotein.
